(19) United States
(12) Patent Application Publication
(10) Pub. No.: US 2008/0262350 A1
(43) Pub. Date: Oct. 23, 2008

(54) Ultrasound Apparatus and Method to Treat an Ischemic Stroke

(75) Inventor: Evan C. Unger, Tucson, AZ (US)

Correspondence Address: DALE F. REGELMAN, QUARLES & BRADY, LLP, ONE SOUTH CHURCH AVENUE AVE, STE. 1700, TUCSON, AZ, 85701-1621, US

(73) Assignee: IMARX THERAPEUTICS, INC., TUCSON, AZ (US)

(21) Appl. No.: 12/094,263

(22) PCT Filed: Mar. 15, 2006
(86) PCT No.: PCT/US2006/009169

§ 371 Date: May 19, 2008

Related U.S. Application Data
(60) Provisional application No. 60/737,980, filed on Nov. 18, 2005.
(60) Provisional application No. 60/738,080, filed on Nov. 18, 2005.

(51) Int. Cl. A61N 7/00; A61B 8/14

(52) U.S. Cl. 600/439; 601/2

(57) ABSTRACT

An apparatus and a method are disclosed to treat a patient sustaining cerebral ischemia or an ischemic stroke in the brain. The method supplies an ultrasound emitting device 100 comprising a plurality of ultrasound transducers (180) and emitting first ultrasound energy by the ultrasound emitting device, where that first ultrasound energy comprises first power. The method locates using the first ultrasound energy an occlusion disposed in one of the patient's cerebral blood vessels. The method then emits second ultrasound energy by the ultrasound emitting device, where that second ultrasound energy comprises second power, where the second power is greater than said first power. The method lyses the occlusion using the second ultrasound energy.

### CROSS REFERENCE TO RELATED APPLICATIONS

This Application claims priority from a U.S. Provisional Application having Ser. No. 60/737,980 filed Nov. 18, 2005, and from a U.S. Provisional Application having Ser. No. 60/738,080 filed Nov. 18, 2005.

### FIELD OF THE INVENTION

Applicants' invention relates to an ultrasound emitting apparatus, and a method using that device to treat ischemic strokes.

### BACKGROUND OF THE INVENTION

Stroke is characterized by the sudden loss of circulation to an area of the brain, resulting in a corresponding loss of neurologic function. Also called cerebrovascular accident or stroke syndrome, stroke is a nonspecific term encompassing a heterogeneous group of pathophysiologic causes, including thrombosis, embolism, and hemorrhage.

Strokes currently are classified as either hemorrhagic or ischemic. Acute ischemic stroke refers to strokes caused by thrombosis or embolism and accounts for 80% of all strokes.

On the macroscopic level, ischemic strokes most often are caused by extracranial embolism or intracranial thrombosis. On the cellular level, any process that disrupts blood flow to a portion of the brain unleashes an ischemic cascade, leading to the death of neurons and cerebral infarction.

Emboli may arise from the heart, the extracranial arteries or, rarely, the right-sided circulation (paradoxical emboli). The sources of cardiogenic emboli include valvular thrombi, resulting from for example in mitral stenosis, endocarditis, prosthetic valves; mural thrombi, resulting from for example myocardial infarction, atrial fibrillation, dilated cardiomyopathy, and the like; and atrial myxomas.

Lacunar infarcts account for 13-20% of all cerebral infarctions and usually involve the small terminal vasculature of the subcortical cerebrum and brainstem. Lacunar infarcts commonly occur in patients with small vessel disease, such as diabetes and hypertension. Small emboli or an in situ process called lipohyalinosis is thought to cause lacunar infarcts. The most common lacunar syndromes include pure motor, pure sensory, and ataxic hemiparetic strokes. By virtue of their small size and well-defined subcortical location, lacunar infarcts do not lead to impairments in cognition, memory, speech, or level of consciousness.

The most common sites of thrombotic occlusion are cerebral artery branch points, especially in the distribution of the internal carotid artery. Arterial stenosis, i.e. turbulent blood flow, atherosclerosis, i.e. ulcerated plaques, and platelet adherence cause the formation of blood clots that either embolize or occlude the artery. Less common causes of thrombosis include polycythemia, sickle cell anemia, protein C deficiency, fibromuscular dysplasia of the cerebral arteries, and prolonged vasoconstriction from migraine headache disorders. Any process that causes dissection of the cerebral arteries also can cause thrombotic stroke, including for example trauma, thoracic aortic dissection, arteritis, and the like. Occasionally, hypoperfusion distal to a stenotic or occluded artery or hypoperfusion of a vulnerable watershed region between 2 cerebral arterial territories can cause ischemic stroke.

Within seconds to minutes of the loss of perfusion to a portion of the brain, an ischemic cascade is unleashed that, if left unchecked, causes a central area of irreversible infarction surrounded by an area of potentially reversible ischemic penumbra. On the cellular level, the ischemic neuron becomes depolarized as ATP is depleted and membrane ion-transport systems fail. The resulting influx of calcium leads to the release of a number of neurotransmitters, including large quantities of glutamate, which in turn activates N-methyl-D-aspartate (NMDA) and other excitatory receptors on other neurons. These neurons then become depolarized, causing further calcium influx, further glutamate release, and local amplification of the initial ischemic insult. This massive calcium influx also activates various degradative enzymes, leading to the destruction of the cell membrane and other essential neuronal structures.

Free radicals, arachidonic acid, and nitric oxide are generated by this process, leading to further neuronal damage. Within hours to days after a stroke, specific genes are activated, leading to the formation of cytokines and other factors that in turn cause further inflammation and microcirculatory compromise. Ultimately, the ischemic penumbra is consumed by these progressive insults, coalescing with the infarcted core, often within hours of the onset of the stroke.

The central goal of therapy in acute ischemic stroke is to preserve the ischemic penumbra. This can be accomplished by limiting the severity of ischemic injury and/or reducing the duration of ischemia, i.e. restoring blood flow to the compromised area.

The timing of restoring cerebral blood flow is critical. Animal and human imaging studies suggest that reperfusion must occur within 3 hours for the ischemic penumbra to be saved. Time also may prove to be a key factor in neuronal protection. What is needed is an apparatus and method that can be used to both locate the situs of the occluded cerebral vessel, and to provide early therapy to lyse that occlusion.

### SUMMARY OF THE INVENTION

Applicants' invention comprises a method for treating a patient sustaining cerebral ischemia or an ischemic stroke in the brain. The method supplies an ultrasound emitting device comprising a plurality of ultrasound transducers, and emitting first ultrasound energy by the ultrasound emitting device, where that first ultrasound energy comprises first power. The method locates using the first ultrasound energy an occlusion disposed in one of the patient's cerebral blood vessels.

The method then emits second ultrasound energy by the ultrasound emitting device, where that second ultrasound energy comprises second power, where the second power is greater than said first power. The method lyses the occlusion using the second ultrasound energy.

### BRIEF DESCRIPTION OF THE DRAWINGS

The invention will be better understood from a reading of the following detailed description taken in conjunction with the drawings in which like reference designators are used to designate like elements, and in which:

FIG. 1A is a perspective view of Applicants' ultrasound emitting device;

FIG. 1B is a side view of the device of FIG. 1A;

FIG. 1C is a perspective view of the device of FIG. 1A showing a housing portion and a bottom portion;

FIG. 2A is a perspective view of an embodiment of Applicants' ultrasound emitting device comprising a bottom portion comprising two offset planar assemblies;

FIG. 2B is a perspective view of the bottom portion of FIG. 2A;

FIG. 2C is a side view of the bottom portion of FIG. 2A;

FIG. 3A is a perspective view of an embodiment of Applicants' ultrasound emitting device comprising a bottom portion comprising four offset planar assemblies;

FIG. 3B is a side view of the bottom portion of FIG. 3A;

FIG. 4A is a block diagram showing one embodiment of Applicants' sound head matrix;

FIG. 4B is a side view of one embodiment of the sound head matrix of FIG. 4A;

FIG. 4C is a side view of a second embodiment of the sound head matrix of FIG. 4A;

FIG. 5A is a block diagram showing a second embodiment of Applicants' sound head matrix;

FIG. 5B is a side view of one embodiment of the sound head matrix of FIG. 5A;

FIG. 5C is a side view of a second embodiment of the sound head matrix of FIG. 5A;

FIG. 6 is a perspective view showing an external controller and power source for Applicants' ultrasound emitting device;

FIG. 7A is a perspective view showing an embodiment of Applicants' ultrasound emitting device comprising an internal controller;

FIG. 7B is a perspective view showing the device of FIG. 7A in combination with an integrated input/output element;

FIG. 8A is a block diagram showing an embodiment of Applicants' ultrasound emitting device which further comprises a diagnostic ultrasound transceiver;

FIG. 8B is a perspective view of the device of FIG. 8A further comprising an internal controller;

FIG. 8C is a perspective view of the device of FIG. 8B further comprising an integrated input/output element;

FIG. 9 is a perspective view of the ultrasound emitting device of FIG. 8B or 8C further comprising a communication port in bidirectional communication with an internal controller;

FIG. 10 is a block diagram showing the ultrasound emitting device of FIG. 9 interconnected with an external computing device;

FIG. 11A is a side view showing Applicants' ultrasound emitting device removeably disposed adjacent a patient's head using a head band apparatus;

FIG. 11B is a side view showing Applicants' ultrasound emitting device removeably disposed adjacent a patient's head using a head frame apparatus;

FIG. 12 is a front view showing two ultrasound emitting devices removeably disposed adjacent a patient's head using either the head band of FIG. 11A or the head frame of FIG. 11B; and

FIG. 13 is a flow chart summarizing the steps of Applicants' method using Applicants' ultrasound emitting apparatus.

### DETAILED DESCRIPTION OF THE PREFERRED EMBODIMENTS

The invention is described in preferred embodiments in the following description with reference to the Figures, in which like numbers represent the same or similar elements. Various embodiments of Applicants' ultrasound emitting apparatus are described herein as comprising sixteen (16) therapeutic ultrasound transducers. This description of Applicants' ultrasound emitting apparatus should not be interpreted to limit Applicants' ultrasound emitting assembly to a total of 16 ultrasound transducers. Rather, Applicants' ultrasound emitting assembly comprises (N) therapeutic ultrasound transducers, wherein (N) is greater than or equal to 1.

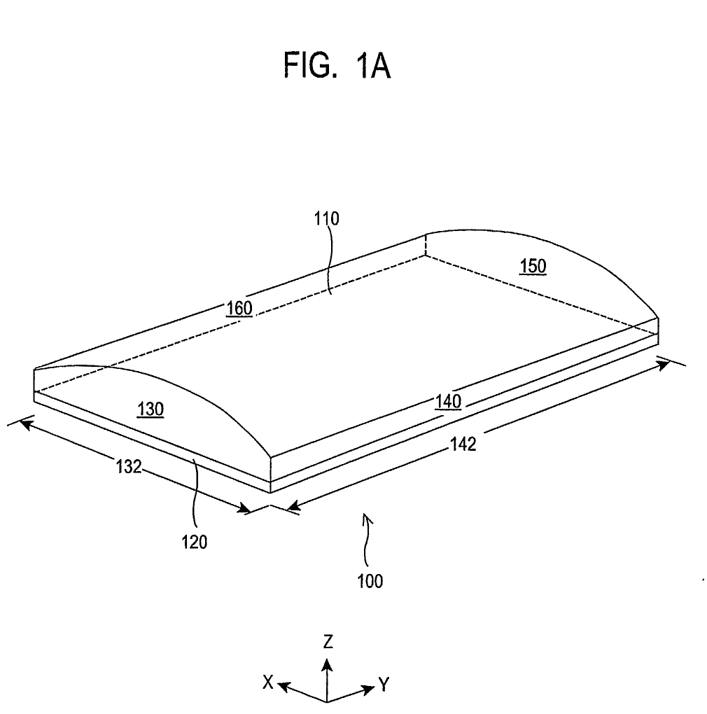

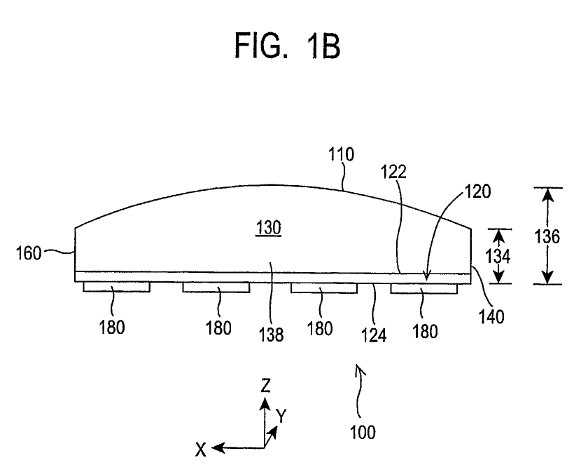

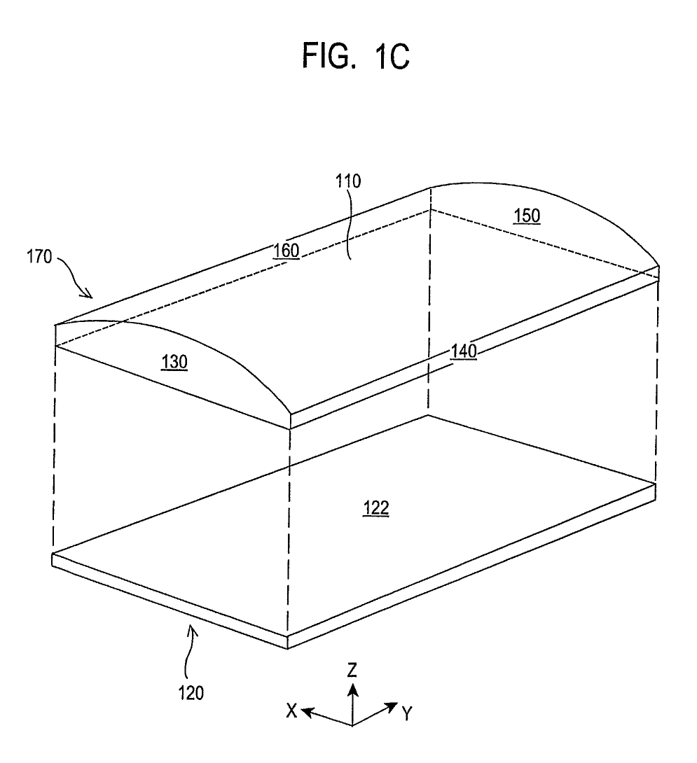

Referring to FIG. 1A, Applicants' ultrasound emitting device 100 comprises a top 110, bottom 120, and sides 130, 140, 150, and 160. In certain embodiments, top 110 and sides 130, 140, 150, and 160, are formed from one or more rigid materials, including wood, metal, plastic, and combinations thereof. In certain embodiments, top 110, and sides 130, 140, 150, and 160, are separately formed, and subsequent attached to one another as shown in FIG. 1 using conventional attachment methods, including welding, sonic welding, plastic welding, adhesive bonding, mechanical attachment, and the like.

Sides 140 and 160 have dimension 142 in the Y direction. In certain embodiments, dimension 142 is between about 10 cm and about 50 cm. Sides 130 and 150 have dimension 132 in the X direction. In certain embodiments, dimension 132 is between about 5 cm and about 25 cm.

FIG. 1B is a side view of apparatus 100. Apparatus 100 includes a plurality of therapeutic ultrasound transducers 180 disposed on, or through, bottom 120. By “therapeutic ultrasound transducer,” Applicants mean a device that is capable of operating at between a 0.1 percent and a 100 percent duty cycle, and that emits therapeutic ultrasound energy. By “therapeutic ultrasound energy,” Applicants mean sound waves having a frequency between about 150 kilohertz and about 10 megahertz or higher, and a power level between about 0.1 watt/cm² and about 30 watts/cm². In certain embodiments, when operated continuously, the output power for each of the plurality of therapeutic ultrasound transducers can as great as about 50 watts. In other embodiments, the output power for each of the plurality of therapeutic ultrasound transducers is between about 6 to about 10 watts.

In the illustrated embodiment of FIG. 1B, sides 130 and 150 vary in dimension along the Z direction, having dimension 134 at the attachment of sides 140 and 160, and dimension 136 at mid point 138. In certain embodiments, dimension 134 is between about 2 cm and about 4 cm. In certain embodiments, dimension 136 is between about 3 cm and about 8 cm. In other embodiments, Applicants' ultrasound emitting device comprises a parallelepiped, i.e. dimension 132 is substantially equal to dimension 134.

Referring to FIG. 1C, in certain embodiments Applicants' ultrasound emitting device 100 comprises housing 170 which includes top 110 and sides 130, 140, 150, and 160. In certain embodiments, housing 170 is integrally formed from one or more metallic materials. In certain embodiments, housing 170 is integrally molded from one or more polymeric materials. In certain embodiments, housing 170 is formed from one or more full density polymeric materials. In certain embodiments, those polymeric materials include polyethylene, polypropylene, polycarbonate, polystyrene, polyvinylchloride, combinations thereof, and the like.

In certain embodiments, those polymeric materials comprise one or more partial-density materials, i.e. one or more cellular materials. In certain embodiments, such cellular materials comprise one or more structural foam materials formed from the group which includes one or more polyurethanes, one or more polystyrenes, and combinations thereof, and the like.

Bottom 120 in combination with housing 170 comprises an enclosure. Bottom 120 includes interior surface 122 and exterior surface 124 (FIG. 1B). In certain embodiments, bottom 120 is formed from metal, one or more polymeric materials, and combinations thereof. In certain embodiments, housing 170 is formed from one or more first polymeric materials and bottom 120 is formed from one or more second polymeric materials, where the one or more first polymeric materials differ from the one or more second polymeric materials.

In certain embodiments, bottom 120 is attached to housing 170 using adhesive bonding. In certain embodiments, bottom 120 is attached to housing 170 using conventional attachment means such as, for example, screws, nuts/bolts, rivets, and the like. In certain embodiments, bottom 120 can be releaseably affixed to housing 170, such that housing 170 can be used with a variety of differing sound head matrix assemblies, as described below.

One or more piezoelectric transducers are disposed on, or through, the exterior surface of the bottom portion of Applicants' device. Each piezoelectric transducer, sometimes referred to as a “sound head,” includes one or more piezoelectric materials. When an alternating current is applied to such a piezoelectric material, deformation occurs wherein the piezoelectric material expands and contracts. Such expansion and contraction crystal produces vibrations, i.e. acoustic waves.

In certain embodiments, Applicants' piezoelectric transducers comprise one or more ceramic materials having pronounced piezoelectric characteristics. In certain embodiments, Applicants' piezoelectric transducers comprise lead zirconate titanate (“PZT”). In other embodiments, Applicants' piezoelectric material comprises lead-magnesium-niobate lead titanate, hereafter referred to for brevity by the acronym PMN-PT. Such PMN-PT materials are described in U.S. Pat. No. 6,737,789.

In certain embodiments, Applicants' piezoelectric materials are formed from a thick-film ink, wherein one or more PZT and/or PMN-PT pastes are mixed with a powdered glass and an organic carrier, which is then printed onto the bottom portion of Applicants' device.

In certain embodiments, the one or more piezoelectric transducers disposed on the exterior of Applicants' device comprise therapeutic ultrasound transducers. By “therapeutic ultrasound transducer,” Applicants mean a device that is capable of operating at between a 0.1 percent and a 100 percent duty cycle, and that emits therapeutic ultrasound energy. By “therapeutic ultrasound energy,” Applicants mean sound waves having a frequency between about 150 kilohertz and about 10 megahertz or higher, and a power level between about 0.1 watt/cm² and about 30 watts/cm². In certain embodiments, when operated continuously, the output power for each of the plurality of therapeutic ultrasound transducers can as great as about 50 watts. In other embodiments, the output power for each of the plurality of therapeutic ultrasound transducers is between about 6 to about 10 watts.

The plurality of therapeutic ultrasound transducers disposed on Applicants' device comprise a sound head matrix. In certain embodiments, Applicants' sound head matrix comprises a plurality of therapeutic ultrasound transducers are arranged in columns and rows. In other embodiments, Applicants' sound head matrix comprises a plurality of therapeutic ultrasound transducers arranged in a pattern comprising concentric circles.

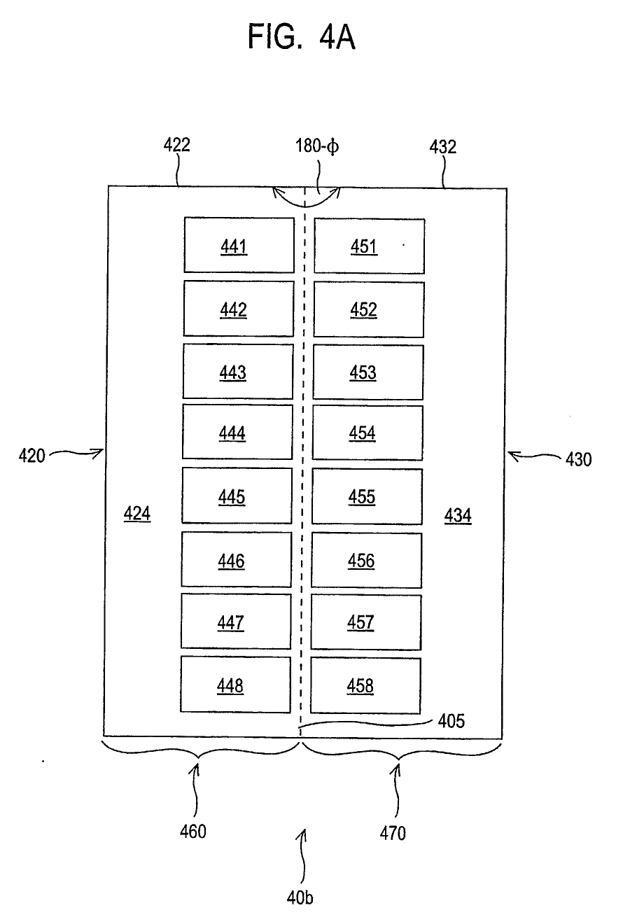

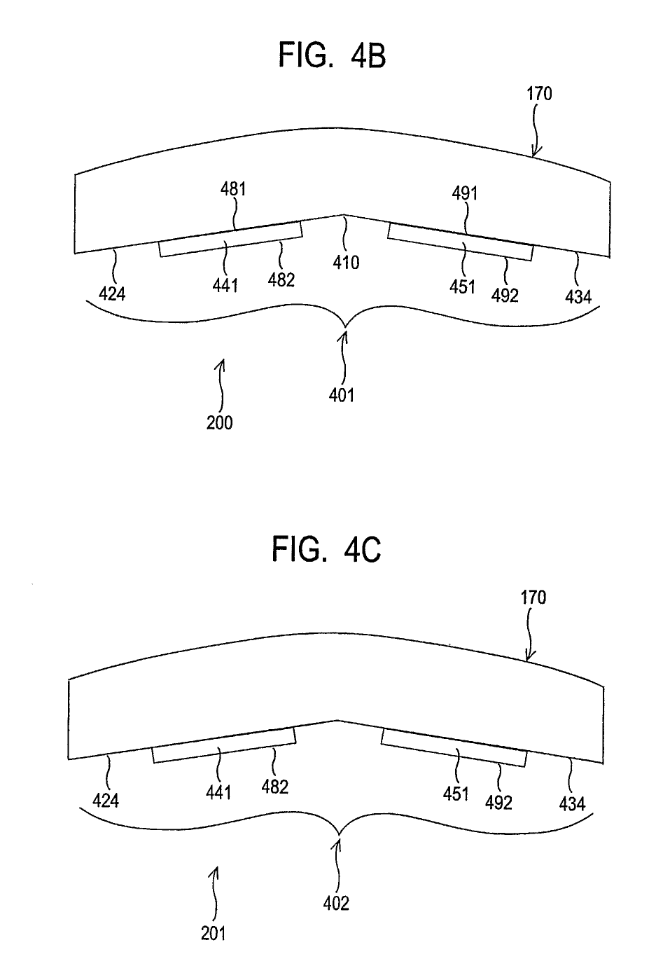

FIG. 4A shows one embodiment of Applicants' sound head matrix. In the illustrated embodiment of FIG. 4A, the sound head matrix comprises sixteen (16) therapeutic ultrasound transducers arranged in two columns of eight (8) transducers. Thus, sound head matrix of FIG. 4A comprises an 8×2 sound head matrix.

Each transducer comprising the sound head matrix of FIG. 4A is disposed on, or through, one of two planar members, either planar member 420 or planar member 430. In certain embodiments, planar member 420 and/or planar member 430 comprises a circuit substrate, wherein one or more electrical circuit components are attached to and/or through that circuit substrate. In certain embodiments, such a circuit substrate comprises what is sometimes referred to as a printed circuit board (“PCB”). In certain embodiments, planar member 420 and/or planar member 430 comprises a single-sided PCB. In certain embodiments, planar member 420 and/or planar member 430 comprises a double-sided PCB. In certain embodiments, planar member 420 and/or planar member 430 comprises a multilayer PCB. In certain embodiments, planar member 420 and/or planar member 430 comprises a metal core, i.e. copper for example, encapsulated with a ceramic coating.

In certain embodiments, planar member 420 and/or planar member 430 comprise a ceramic material. In certain embodiments, planar member 420 and/or planar member 430 comprise aluminum oxide. In certain embodiments, planar member 420 and/or planar member 430 comprise beryllium oxide.

In embodiments wherein housing 170 comprises one or more metallic components, and wherein planar members 420 and/or 430 comprise a ceramic material and/or a ceramic material encapsulating a copper core, planar members 420 and/or 430 conduct heat generated by the plurality of ultrasound emitters from the core of Applicants' device to the metallic housing, i.e. the circuit substrates in combination with the housing, comprise, inter alia, an integrated heat sink assembly which continuously dissipates heat from Applicants' device to the environment.

Planar member 420 is continuously attached to planar member 430 along common edge, i.e. seam, 405. Transducers 441, 442, 443, 444, 445, 446, 447, and 448, are disposed on, or through, surface 424 of planar member 420. Transducers 441, 442, 443, 444, 445, 446, 447, and 448, in combination with planar member 420, comprises planar assembly 460. Transducers 451, 452, 453, 454, 455, 456, 457, and 458, are disposed on, or through, surface 434 of planar member 430. Transducers 451, 452, 453, 454, 455, 456, 457, and 458, in combination with planar member 430, comprises planar assembly 470.

Planar assembly 460 in combination with planar assembly 470 comprises sound head matrix assembly 401. In certain embodiments, sound head matrix assembly 401 comprises a flat structure. In other embodiments, sound head matrix assembly 401 is not flat, i.e. the dihedral angle formed by the intersection of assemblies 460 and 470 does not equal 180 degrees.

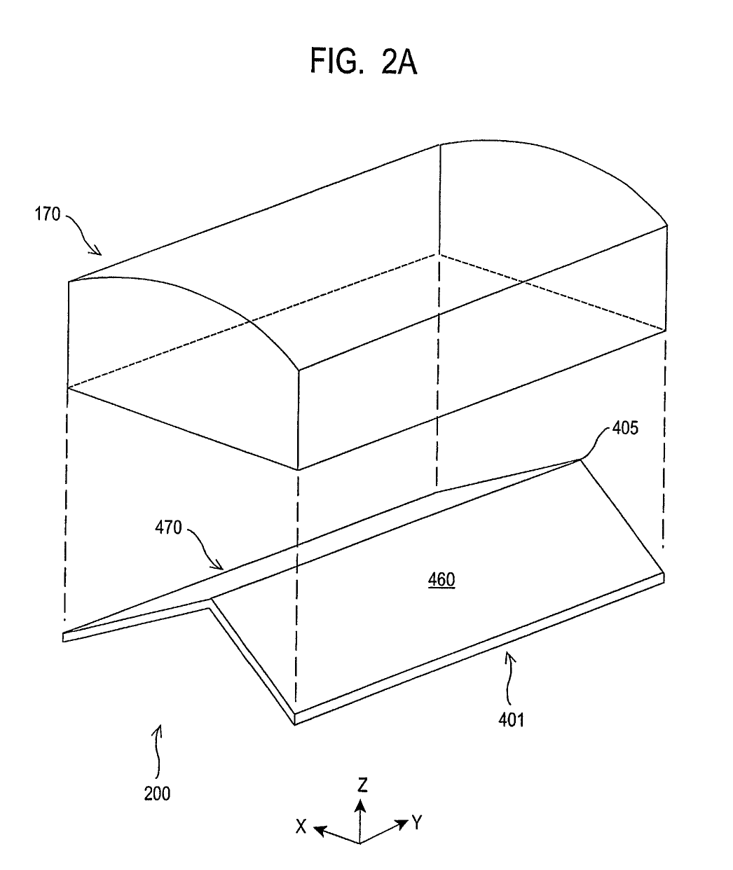

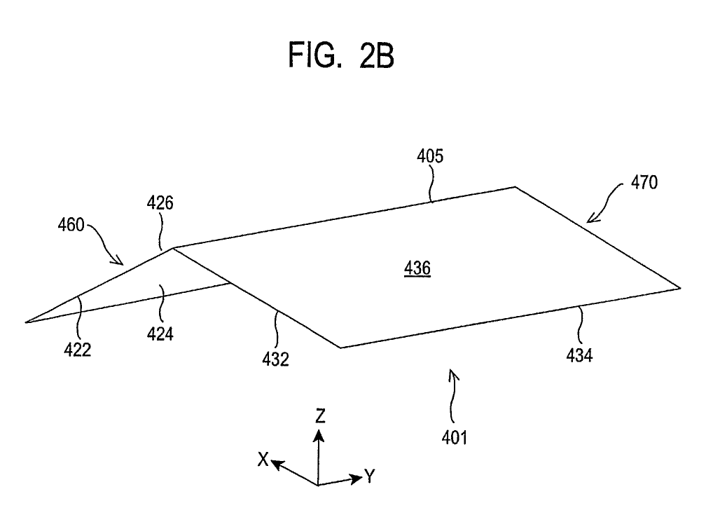

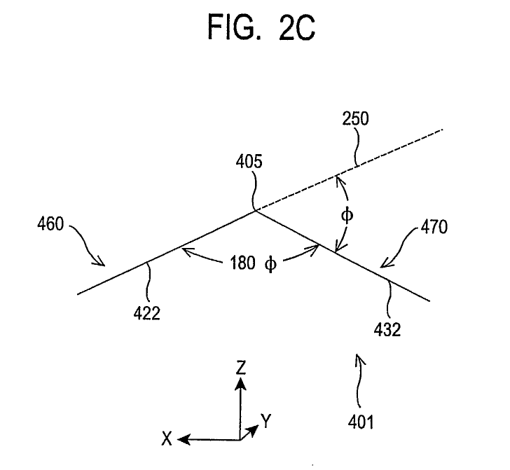

Referring to FIG. 2A, device 200 includes housing 170 (FIG. 1C) in combination with an “offset” embodiment of sound head matrix assembly 401. As described above, sound head matrix assembly 401 includes planar assembly 460 in combination with planar assembly 470, where planar assembly 460 is continuously joined to planar assembly 470 along common edge 405. Planar assembly 460 lies in a first plane, and planar assembly 470 lies in a second plane. That first plane intersects the second plane along common edge 405 to form an interior dihedral angle, as defined herein, less than 180 degrees.

Referring now to FIGS. 2A, 2B, and 2C, planar assembly 460 includes edge 422. Planar assembly 470 includes edge 432. Edge 422 meets edge 432 at seam 405. Dotted line 250 represents the extension of edge 422 past seam 405. As shown in FIG. 2C, angle Φ represents the angle formed between edge 432 and extension line 250. For purposes of this Application, planar assembly 460 is “offset” from planar assembly 470 by angle Φ. As those skilled in the art will appreciate, the interior dihedral angle, in degrees, formed by the intersection of planar assembly 460 and planar assembly 470 is 180-ΦD.

In certain embodiments, angle Φ is between about 5 degrees and about 25 degrees. In certain embodiments, angle Φ is between about 10 degrees and about 20 degrees. In certain embodiments, angle Φ is about 13 degrees.

As those skilled in the art will appreciate, the interior dihedral angle formed by planar assembly 460 and planar assembly 470 is inversely proportional to the offset angle Φ. Therefore, as Φ increases from 0 degrees, the dihedral angle decreases from 180 degrees. Thus, where planar assembly 460 is “offset” from planar assembly 470 by, for example, 15 degrees, then the interior dihedral angle formed by planar assembly 460 and planar assembly 470 is 165 degrees.

FIG. 4B shows a side view of apparatus 200 which includes housing 170 in combination with an offset sound head matrix assembly 401. Transducer 441 (FIGS. 4A, 4B, 4C) comprises a first side 481 and an opposing second side 482. Transducer 451 (FIGS. 4A, 4B, 4C) includes a first side 491 and an opposing second side 492. In the illustrated embodiment of FIG. 4B, side 481 of transducer 441 is disposed on surface 424 of planar member 420, and side 491 of transducer 451 is disposed on surface 434 of planar member 430. As those skilled in the art will appreciate, transducers 441 may include one or more leads which extend through holes, i.e. vias, drilled through planar member 420. In other embodiments, transducer 441 comprises what is sometimes called a “surface mounted” device, wherein that surface mounted device is attached to a solder pad disposed on surface 424.

FIG. 4C shows a side view of apparatus 201 which includes housing 170 in combination with an offset sound head matrix assembly 402. Sound head matrix assembly 402 is identical to sound head matrix assembly 401 except that each of the plurality of therapeutic ultrasound transducers extends through a planar member rather than being disposed on that planar member. For example in the illustrated embodiment of FIG. 4C, transducer 441 is disposed through planar member 420 such that surface 482 of transducer 441 is flush with surface 424 of planar assembly 460. Similarly in this embodiment, transducer 451 is disposed through planar member 430 such that surface 492 of transducer 451 is flush with surface 434 of planar assembly 470.

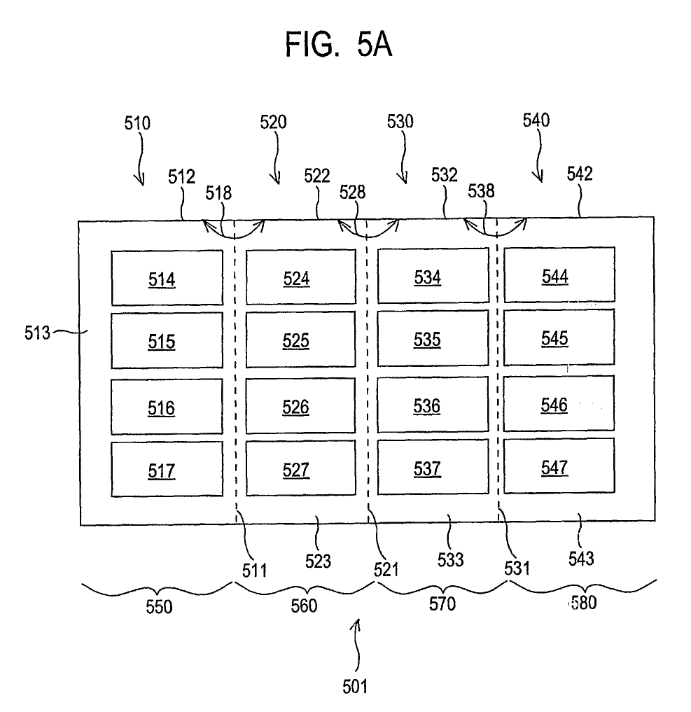

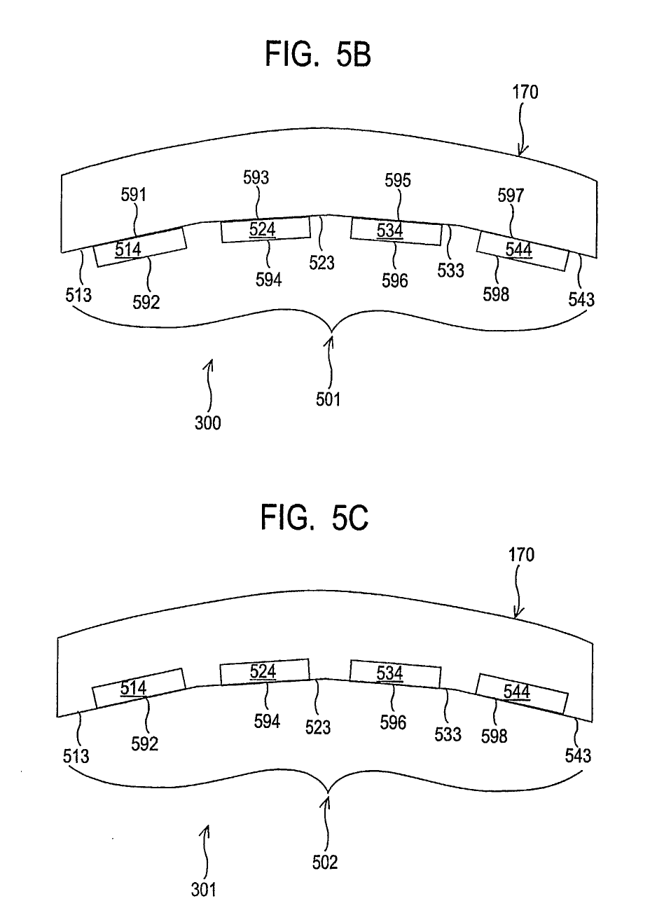

FIG. 5A shows another embodiment of Applicants' sound head matrix. In the illustrated embodiment of FIG. 5A, the sound head matrix comprises sixteen (16) therapeutic ultrasound transducers arranged in four columns of four transducers. Thus, sound head matrix of FIG. 5A comprises an 4×4 sound head matrix.

Each transducer comprising the sound head matrix of FIG. 5A is disposed on, or through, one of four planar members, namely planar member 510, or planar member 520, or planar member 530, or planar member 540. Planar member 510 is continuously attached to planar member 520 along common edge 511. Transducers 514, 515, 516, and 517, are disposed on, or through, surface 513 of planar member 510. Transducers 514, 515, 516, and 517, in combination with planar member 510, comprise planar assembly 550. Angle 518 comprises the interior dihedral angle formed by the intersection of planar member 510 with planar member 520.

In certain embodiments, angle 518 is about 180 degrees. In these embodiments, planar member 510 is not offset from planar member 520, i.e. planar member 510 in combination with planar member 520 comprises a flat assembly. In other embodiments, angle 518 is less than 180 degrees, i.e. planar member 510 is offset from planar member 520.

In certain embodiments, planar members 510 and 520 are integrally formed to include angle 518. In other embodiments, planar members 510 and 520 are individually formed, and subsequently attached using conventional attachment methods.

Planar member 520 is continuously attached to planar member 530 along common edge 521. Transducers 524, 525, 526, and 527, are disposed on, or through, surface 523 of planar member 520. Transducers 524, 525, 526, and 527, in combination with planar member 520, comprise planar assembly 560. Angle 528 comprises the interior dihedral angle formed by the intersection of planar member 520 with planar member 530.

In certain embodiments, angle 528 is about 180 degrees. In these embodiments, planar member 520 is not offset from planar member 530, i.e. planar member 520 in combination with planar member 530 comprises a flat assembly. In other embodiments, angle 528 is less than 180 degrees, i.e. planar member 520 is offset from planar member 530.

In certain embodiments, planar members 520 and 530 are integrally formed to include angle 528. In other embodiments, planar members 520 and 530 are individually formed, and subsequently attached using conventional attachment methods.

Planar member 530 is continuously attached to planar member 540 along common edge 531. Transducers 534, 535, 536, and 537, are disposed on, or through, surface 533 of planar member 530. Transducers 534, 535, 536, and 537, in combination with planar member 530, comprise planar assembly 570. Angle 538 comprises the interior dihedral angle formed by the intersection of planar member 530 with planar member 540.

In certain embodiments, angle 538 is about 180 degrees. In these embodiments, planar member 530 is not offset from planar member 540, i.e. planar member 530 in combination with planar member 540 comprises a flat assembly. In other embodiments, angle 538 is less than 180 degrees, i.e. planar member 530 is offset from planar member 540.

In certain embodiments, planar members 530 and 540 are integrally formed to include angle 538. In other embodiments, planar members 530 and 540 are individually formed, and subsequently attached using conventional attachment methods.

Transducers 544, 545, 546, and 547, are disposed on, or through, surface 543 of planar member 530. Transducers 544, 545, 546, and 547, in combination with planar member 540, comprise planar assembly 580.

Planar assemblies 550, 560, 570, and 580, in combination, comprise sound head matrix assembly 501. In certain embodiments, sound head matrix assembly 501 comprises a flat structure. In other embodiments, sound head matrix assembly 501 is not flat.

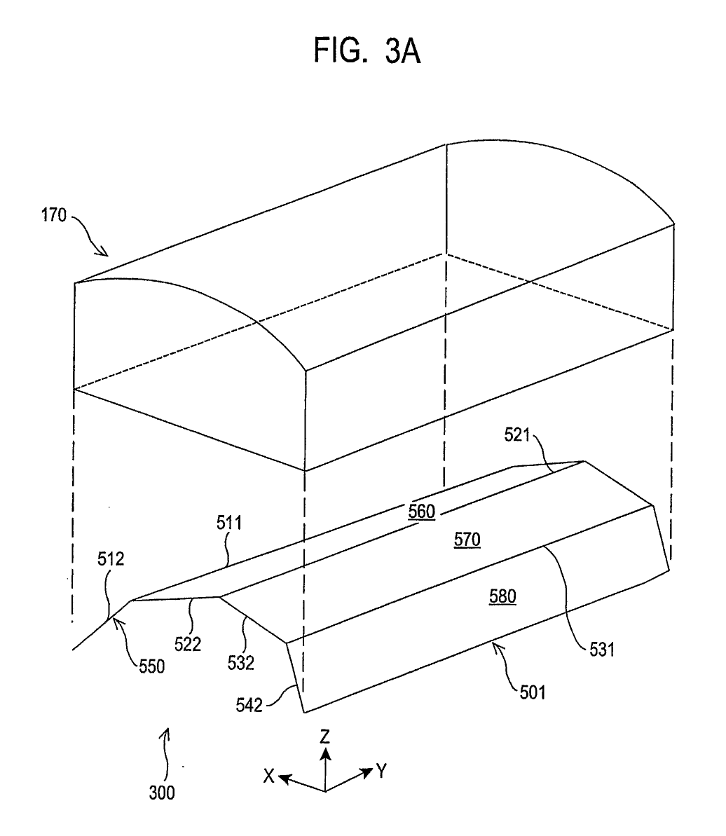

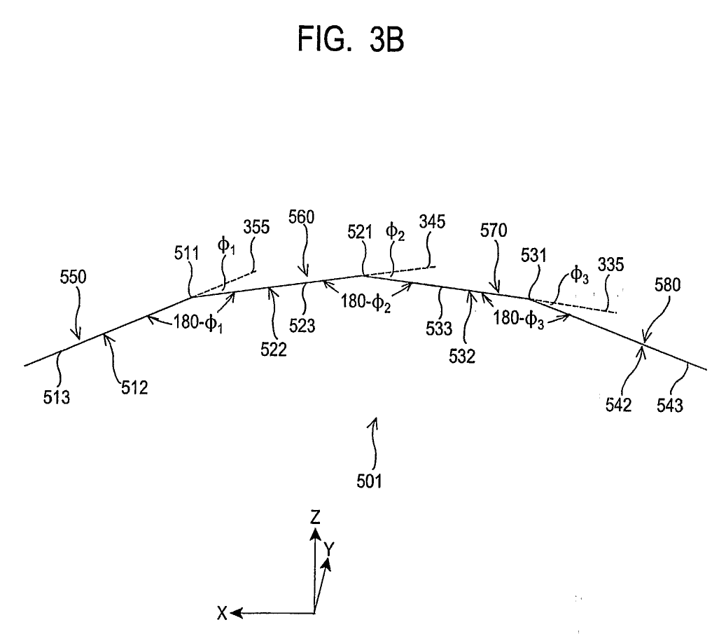

Referring to FIGS. 3A and 3B, Applicants' ultrasonic emitting apparatus 300 includes housing 170 (FIG. 1C) in combination with sound head matrix assembly 501 (FIGS. 3A, 3B, 5A, 5B). Edge 512 of planar assembly 550 meets edge 522 of planar assembly 560 along seam 511. Dotted line 355 represents the extension of edge 512 past seam 511. As shown in FIG. 3B, angle Φ1 represents the angle formed between edge 522 and extension line 335. For purposes of this Application, planar assembly 550 is “offset” from planar assembly 560, where the offset angle is angle Φ1. As those skilled in the art will appreciate, the interior dihedral angle, in degrees, formed by the intersection of planar assembly 550 and planar assembly 560 is 180-Φ1. By “interior dihedral angle,” Applicants' mean the angle formed between surface 513 and surface 523.

In certain embodiments, angle Φ1 is between about 5 degrees and about 25 degrees. In certain embodiments, angle Φ1 is between about 8 degrees and about 15 degrees. In certain embodiments, angle Φ1 is about 13 degrees.

Edge 522 of planar assembly 560 meets edge 532 of planar assembly 570 along seam 521. Dotted line 345 represents the extension of edge 522 past seam 521. As shown in FIG. 3B, angle Φ2 represents the angle formed between edge 532 and extension line 345. For purposes of this Application, planar assembly 560 is “offset” from planar assembly 570, where the offset angle is angle Φ2. As those skilled in the art will appreciate, the interior dihedral angle, in degrees, formed by the intersection of planar assembly 560 and planar assembly 570 is 180-Φ1. By “interior dihedral angle,” Applicants' mean the angle formed between surface 523 and surface 533.

In certain embodiments, angle Φ2 is between about 5 degrees and about 25 degrees. In certain embodiments, angle Φ2 is between about 8 degrees and about 15 degrees. In certain embodiments, angle Φ2 is about 10 degrees.

Edge 532 of planar assembly 570 meets edge 542 of planar assembly 570 along seam 531. Dotted line 335 represents the extension of edge 532 past seam 531. As shown in FIG. 3B, angle Φ3 represents the angle formed between edge 542 and extension line 335. For purposes of this Application, planar assembly 570 is “offset” from planar assembly 580, where the offset angle is angle Φ3. As those skilled in the art will appreciate, the interior dihedral angle, in degrees, formed by the intersection of planar assembly 570 and planar assembly 580 is 180-Φ1. By “interior dihedral angle,” Applicants' mean the angle formed between surface 533 and surface 543.

In certain embodiments, angle Φ3 is between about 5 degrees and about 25 degrees. In certain embodiments, angle Φ3 is between about 8 degrees and about 15 degrees. In certain embodiments, angle Φ3 is about 13 degrees.

In certain embodiments, two or more of offset angles Φ1, Φ2, and/or Φ3, are substantially the same. By “substantially the same,” Applicants means within about plus or minus ten percent or less. In other embodiments, two or more of offset angles Φ1, Φ2, and/or Φ3, differ.

FIG. 5B shows a side view of apparatus 300 which includes housing 170 in combination with a multiply offset sound head matrix assembly 501. Transducers 514, 524, 534, and 544, each comprise a first side 591, 593, 595, and 597, respectively, and an opposing second side 592, 594, 596, and 598, respectively.

In the illustrated embodiment of FIG. 5B, side 591 of transducer 441, and side 593 of transducer 524, and side 595 of transducer 534, and side 597 of transducer 544, respectively, are disposed on surface 513 of planar assembly 550, surface 523 of planar assembly 560, surface 533 of planar assembly 570, and surface 543 of planar assembly 580, respectively. Transducers 515, 516, 517, 525, 526, 527, 535, 536, 537, 545, 546, and 547, are similarly attached to their respective planar assemblies.

As those skilled in the art will appreciate, the plurality of transducers comprising sound head matrix assembly 501 may include one or more leads which extend through holes, i.e. vias, drilled through one of the four planar assemblies. In other embodiments, the plurality of transducers comprising sound head matrix 501 each comprise what is sometimes called a “surface mounted” device, wherein that surface mounted device is attached to a solder pad disposed on surface 513, or surface 523, or surface 533, or surface 443.

FIG. 5C shows a side view of apparatus 301 which includes housing 170 in combination with an offset sound head matrix assembly 502. Sound head matrix assembly 502 is identical to sound head matrix assembly 501 except that each of the plurality of therapeutic ultrasound transducers extends through a planar assembly rather than being disposed on the exterior surface of that planar assembly. For example in the illustrated embodiment of FIG. 5C, transducers 514, 524, 534, and 544, respectively, are disposed through planar assembly 550, planar assembly 560, planar assembly 570, and planar assembly 580, respectively, such that surface 592 of transducer 514 is flush with surface 513 of planar assembly 550, and, such that surface 594 of transducer 524 is flush with surface 523 of planar assembly 560, and such that surface 596 of transducer 534 is flush with surface 533 of planar assembly 570, and such that surface 598 of transducer 544 is flush with surface 543 of planar assembly 580.

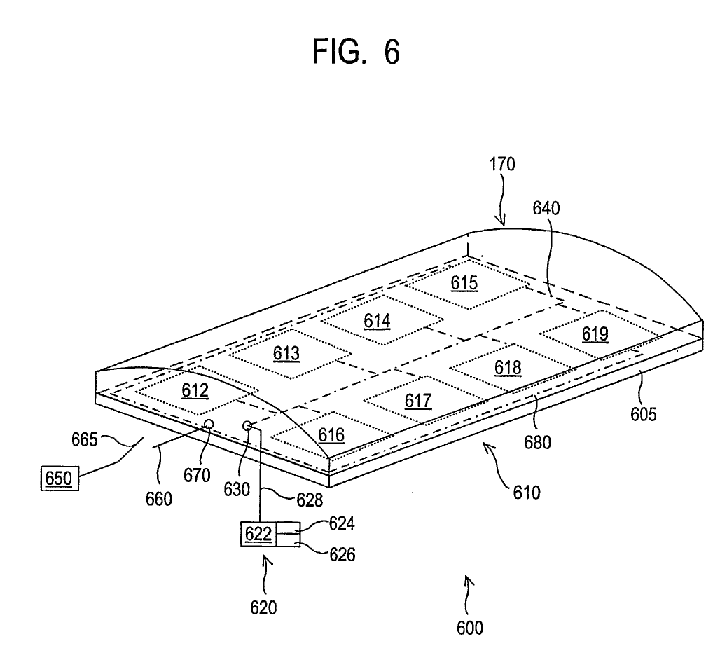

FIG. 6 shows one embodiment of Applicants' therapeutic ultrasound apparatus 600. Apparatus 600 includes ultrasonic emitting device 610, external controller 620, and power source 650. Power source 650 provides power to device 610 by power cable 660. In certain embodiments, Applicants' system 600 includes power switch 665. In the illustrated embodiment of FIG. 6 power switch 665 is disposed in power cable 660. In other embodiments, switch 665 is disposed on power source 650. In other embodiments, switch 665 is disposed on the outer surface of device 610. Power switch 665 can comprise any suitable power switching device, and may take the form of, for example, a rocker switch, a toggle switch, a push to operate switch, and the like.

Device 610 includes housing 170 and sound head matrix assembly 605. In the illustrated embodiment of FIG. 6, Applicants' sound head matrix assembly 605 comprises a 4×2 sound head matrix. As a general matter, Applicants' sound head matrix assembly 605 comprises a Y×Z sound head matrix, wherein Y represents the number of transducers in a column, and wherein Z represents the number of columns, wherein Y is greater than or equal to 1, and less than or equal to about 10, and wherein Z is greater than or equal to 1 and less than or equal to about 6.

For example in certain embodiments, Applicants' ultrasonic device 610 comprises an 8×2 sound head matrix, such as the sound head matrix recited in FIG. 4A. In certain embodiments, Applicants' ultrasonic device 610 comprises a 4×4 sound head matrix, such as the sound head matrix recited in FIG. 5A.

In the illustrated embodiment of FIG. 6, Applicants' sound head matrix assembly is substantially flat. In other embodiments, Applicants' sound head matrix assembly comprises (N) offset planar assemblies, wherein (N) is greater than or equal to 2 and less than or equal to about 6.

For example, in certain embodiments, Applicants' ultrasonic device 610 comprises offset sound head matrix assembly 401 (FIGS. 2A, 3A, 4A, 4B)), where that sound head matrix assembly comprises a Y×2 sound head matrix. In other embodiments, Applicants' ultrasonic device 610 comprises offset sound head matrix assembly 402 (FIG. 4C), where that sound head matrix assembly comprises a Y×2 sound head matrix. In other embodiments, Applicants' ultrasonic device 610 comprises offset sound head matrix assembly 501 (FIGS. 5A, 5B), where that sound head matrix assembly comprises a Y×4 sound head matrix. In other embodiments, Applicants' ultrasonic device 610 comprises offset sound head matrix assembly 502 (FIG. 5C), where that sound head matrix assembly comprises a Y×4 sound head matrix.

Controller 620 is interconnected with device 610 by communication link 628. In certain embodiments, communication link 628 is selected from the group which includes a serial interconnection, such as RS-232 or RS-422, an ethernet interconnection, a SCSI interconnection, a Fibre Channel interconnection, an ESCON interconnection, a FICON interconnection, a Local Area Network (LAN), a private Wide Area Network (WAN), a public wide area network, Storage Area Network (SAN), Transmission Control Protocol/Internet Protocol (TCP/IP), the Internet, and combinations thereof.

In certain embodiments, controller 620 wirelessly communicates with device 610 using Bluetooth-compliant emissions at about 2.4 GHz. In certain embodiments, communication link 628 is compliant with one or more of the embodiments of IEEE Specification 802.11 (collectively the “IEEE Specification”). As those skilled in the art will appreciate, the IEEE Specification comprises a family of specifications developed by the IEEE for wireless LAN technology.

The IEEE Specification specifies an over-the-air interface between a wireless client, such as for example projector 100, and a base station or between two wireless clients. The IEEE accepted the IEEE Specification in 1997. There are several specifications in the 802.11 family, including (i) specification 802.11 which applies to wireless LANs and provides 1 or 2 Mbps transmission in the 2.4 GHz band using either frequency hopping spread spectrum (FHSS) or direct sequence spread spectrum (DSSS); (ii) specification 802.11a which comprises an extension to 802.11 that applies to wireless LANs and provides up to 54 Mbps in the 5 GHz band using an orthogonal frequency division multiplexing encoding scheme rather than FHSS or DSSS; (iii) specification 802.11b, sometimes referred to as 802.11 High Rate or Wi-Fi, which comprises an extension to 802.11 that applies to wireless LANS and provides up to about 11 Mbps transmission in the 2.4 GHz band; and/or (iv) specification 802.11g which applies to wireless LANs and provides 20+ Mbps in the 2.4 GHz band.

Communication link 628 can be releaseably attached to coupling 630 disposed on housing 170. Coupling 630 is interconnected with control bus 640. Control bus 640 is interconnected to each transducer comprising Applicants' sound head matrix assembly 610.

In certain embodiments, controller 620 provides control signals to device 610 wirelessly. In these wireless embodiments, communication link 628 comprises a first antenna coupled to controller 620 and coupling 630 comprises a second antenna coupled to communication bus 640.

Controller 620 includes processor 622, memory 624, and device microcode 626. In certain embodiments, memory 624 comprises one or more nonvolatile memory devices. In certain embodiments, such nonvolatile memory is selected from the group which includes one or more EEPROMs (Electrically Erasable Programmable Read Only Memory), one or more flash PROMs (Programmable Read Only Memory), battery backup RAM, hard disk drive, combinations thereof, and the like.

In certain embodiments, microcode 626 is stored in memory 624. Device microcode 626 comprises instructions residing in memory, such as for example memory 624, where those instructions are executed by processor 622 to implement the selected operational mode for the plurality of transducers comprising Applicants' sound head matrix assembly.

For example, where Applicants' ultrasound emitting device comprises (N) therapeutic ultrasound transducers processor 622 provides the (i)th signal to the (i)th therapeutic ultrasound transducer causing that (i)th therapeutic ultrasound transducer to emit the (i)th therapeutic ultrasound energy comprising the (i)th frequency and the (i)th phase, wherein (i) is greater than or equal to 1 and less than or equal to (N).

In certain embodiments, device microcode 626 comprises instructions residing in memory, such as for example memory 624, where those instructions are executed by processor 622 to cause each of the plurality of therapeutic ultrasound transducers comprising Applicants' sound head matrix assembly 605 to operate continuously. In other embodiments, device microcode 626 comprises instructions residing in memory, such as for example memory 624, where those instructions are executed by processor 622 to cause each of the plurality of therapeutic ultrasound transducers comprising Applicants' sound head matrix assembly 605 to operate discontinuously.

As a general matter, such discontinuous operation modes include embodiments wherein each of the plurality of therapeutic ultrasound transducers comprising Applicants' sound head matrix assembly 605 operates on a duty cycle from about 0.1 percent to 100 percent. In certain embodiments, such discontinuous operation modes include embodiments wherein each of the plurality of therapeutic ultrasound transducers comprising Applicants' sound head matrix assembly 605 operates on a duty cycle selected from the group comprising a 20 percent duty cycle, a 40 percent duty cycle, a 60 percent duty cycle, and an 80 percent duty cycle.

In certain of these discontinuous operational modes, each of the plurality of therapeutic ultrasound transducers comprising Applicants' sound head matrix assembly 605 operates independently of any of the other transducer, i.e. each transducer is alternately turned on and off randomly. In other embodiments, an entire column of transducers operates at the same time, while transducers comprising other columns do not operate. In other embodiments, an entire row of transducers operates at the same time, while transducers comprising other rows do not operate.

In certain embodiments of Applicants' method using Applicants' ultrasound emitting apparatus, combinations of frequencies from differing transducers are employed to effectively treat complex structures. Various frequencies and combinations of frequencies may be desirable in particular circumstances to both avoid standing waves with excessively concentrated energy deposition in particular locations and to provide more uniform distribution of the energy at therapeutic levels. For example, lower frequency acoustic waves, such as 40 kHz, may be better dispersed by refraction of the beam when directed through a small opening in a bone structure. The lower frequency provides longer range and better coverage than higher frequencies. In relation to the skull in particular, lower frequencies also pass through bone more efficiently than higher frequencies.

In general, acoustic waves at higher frequencies penetrate less well, degrade faster, and are much shorter than lower frequency waves. As a result, use of higher frequency waves avoids a problem of low frequency waves that may match the scale of anatomical structures, and thereby, form detrimental large standing waves in such anatomical structures. Also, higher frequencies do not disperse to the same extent as lower frequencies and may therefore be more effective as a straight beam, either aimed at a target or swept through a range of vectors to cover a volume. In addition, higher frequencies, above 500 kHz and particularly between 500 kHz and 2 MHz, are helpful in avoiding unanticipated peaks in the energy deposition pattern and standing waves.

In addition, in certain embodiments the frequency and/or phase of the acoustic waves produced by the plurality of therapeutic ultrasound transducers comprising Applicants' sound head matrix is variable. In certain embodiments, each of the plurality of transducers emits acoustic waves having substantially the same frequency, but with differing phases. In other embodiments, each of the plurality of transducers emits a pattern of modulated acoustic waves wherein the frequency and/or phase of the acoustic waves emitted by each of those transducers is continuously changed from an initial, i.e. beginning, frequency and phase, through a final, i.e. ending frequency and phase. In certain embodiments, each of the transducers comprising Applicants' sound head matrix operates using a different frequency modulation pattern and/or a different phase modulation pattern.

In certain embodiments, the frequency of one or more of Applicants' therapeutic transducers initial emit acoustic waves comprising a low frequency, i.e. 250 KHz and sweep through intervening frequencies to an ending frequency of about 2 MHZ. In certain embodiments, each of the therapeutic transducers using this “low to high” frequency modulation pattern generates acoustic waves having a different phase than the waves emitted from the other “low to high” transducers. Other transducers comprising Applicants' sound head matrix initially emit acoustic waves comprising a high frequency, i.e. 2 MHZ, and sweep through intervening frequencies to an ending frequency of about 250 KHz. In certain embodiments, each of the therapeutic transducers using this “high to low” frequency modulation pattern generates acoustic waves having a different phase than the waves emitted from the other “high to low” transducers.

As those skilled in the art will appreciate, interference occurs when two or more ultrasound waves intersect. The waves may be produced directly from an ultrasound transducer or from a reflection from an anatomical structure, such as the surface of the head. Interference may be either constructive or destructive in nature depending upon the relative phase and amplitudes of the combining waves.

Such interference may be constructive or destructive. Constructive interference occurs when waves having about the same phase intersect with a resulting additive effect regarding the composite energy produced. Destructive interference results when waves having opposing phases intersect with a resulting canceling effect.

If the interference is destructive, i.e. canceling, then when microbubbles are used as the lysing agent, the microbubbles may not expand and contract sufficiently to produce the desired therapeutic effect. In certain embodiments, the ultrasound frequency and phase from one or more therapeutic ultrasound transducers comprising Applicants' sound head matrix is modulated by controller 620 with the result that any interference pattern(s) will be constantly shifting in position, thereby insuring uniform coverage of the targeted anatomical portion of the patients' cerebral anatomy. In addition, the interference pattern of nodes and anti-nodes created thereby is not static but travels through the targeted tissue. Moreover, the frequencies of the acoustic signals are selected to avoid standing waves from resonance of the anatomical portion into which the acoustics signals are delivered.

In certain embodiments, controller 620 comprises a computer, which in addition to memory 624 and microcode 624, further includes one or more input devices, such as for example a keyboard, a mouse, a pointing device, and the like. In certain embodiments, that computer further includes one or more output devices, such as for example one or more monitors, one or more printers, and the like.

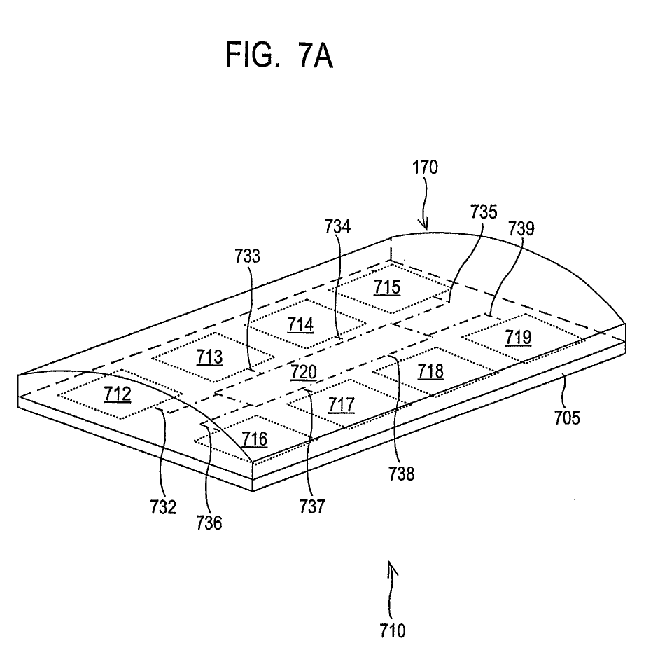

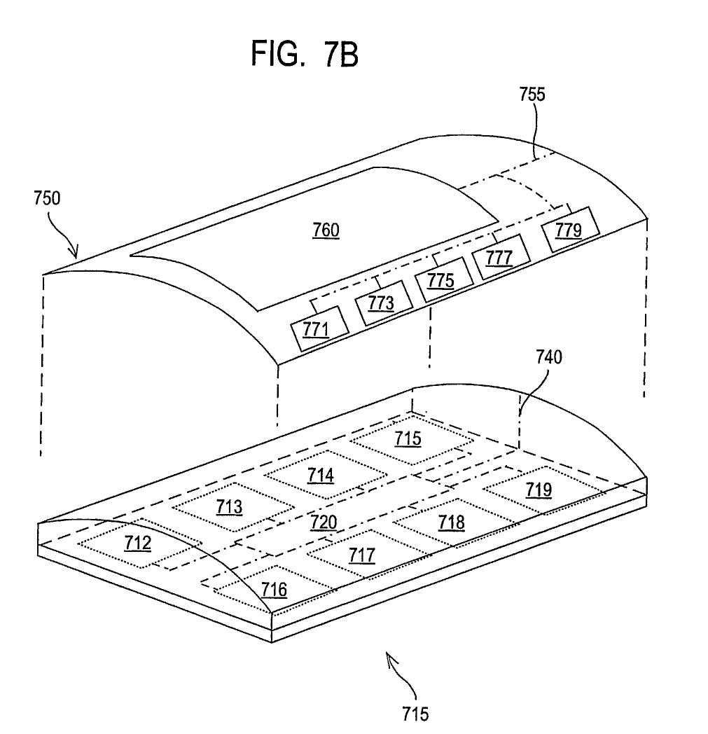

In certain embodiments of Applicants' apparatus, the external control circuitry of FIG. 6, i.e. controller 620, is disposed within Applicants' ultrasonic device. Referring to FIG. 7A, device 710 includes the elements of device 610 in combination with controller 720. For clarity of illustration, FIG. 7 does not include power source 650, power cable 660, or power bus 605. Controller 720 comprises processor 622, memory 624, and microcode 626.

Applicants' ultrasonic device 710 includes controller 720 which is interconnected to each of a plurality of therapeutic ultrasound transducers 712, 713, 714, 715, 716, 717, 718, and 719, via communication links 732, 733, 734, 735, 736, 737, 738, and 739, respectively.

For further clarity of illustration, the illustrated embodiment of FIG. 7A includes 4×2 sound head matrix assembly 705. As a general matter, sound head matrix assembly 705 comprises a Y×Z sound head matrix, where that Y×Z sound head matrix is described above, and where that Y×Z sound head matrix may comprise a substantially flat assembly, or that Y×Z sound head matrix assembly may comprise (N) offset planar assemblies. In certain embodiments, controller 720 comprises an application specific integrated circuit, i.e. an “ASIC,” which integrates the functions of processor 622, memory 624, and microcode 626.

Referring now to FIG. 7B, Applicants' ultrasonic device 715 includes the elements of device 710 (FIG. 7A) in combination with integrated information input/output (“I/O”) device 750. In the illustrated embodiment of FIG. 7B, I/O device 750 includes a visual display device 760 and a plurality of input device/touch screens 771, 773, 775, 777, and 779. In certain embodiments, visual display device 760 comprises an LCD device. I/O device 750 communicates with controller 720 via communication links 740 and 755.

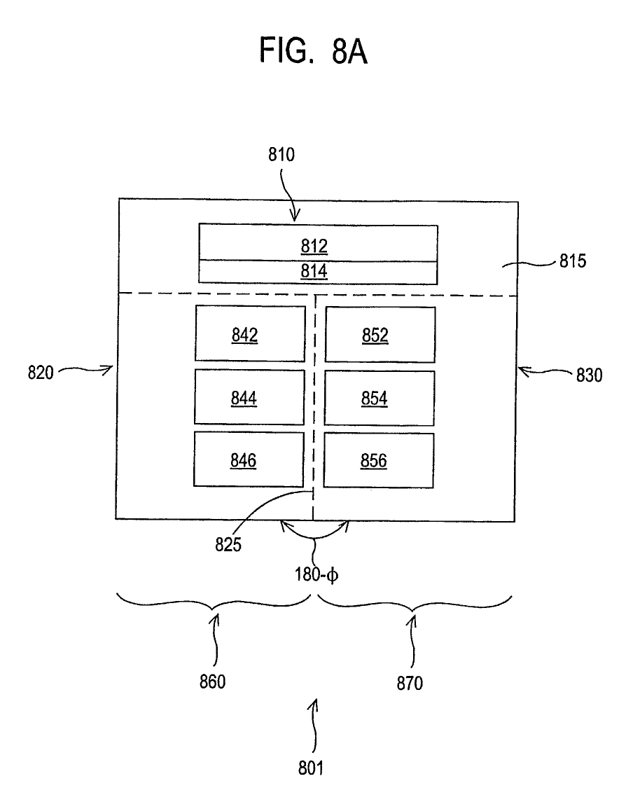

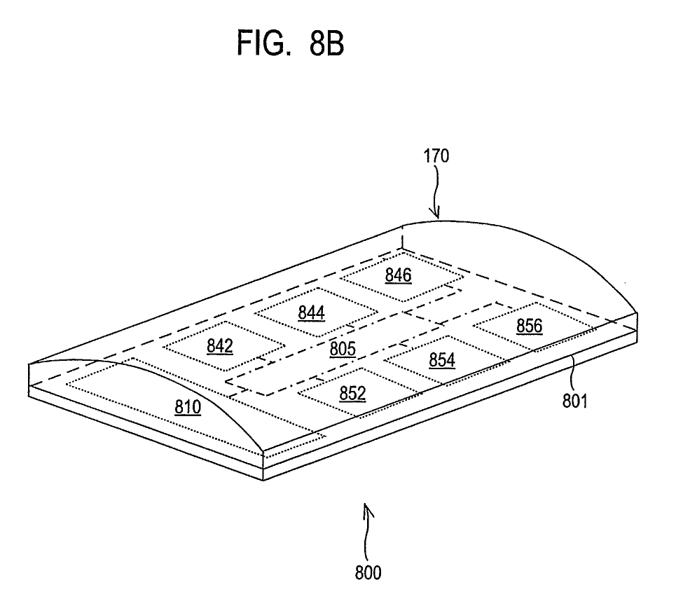

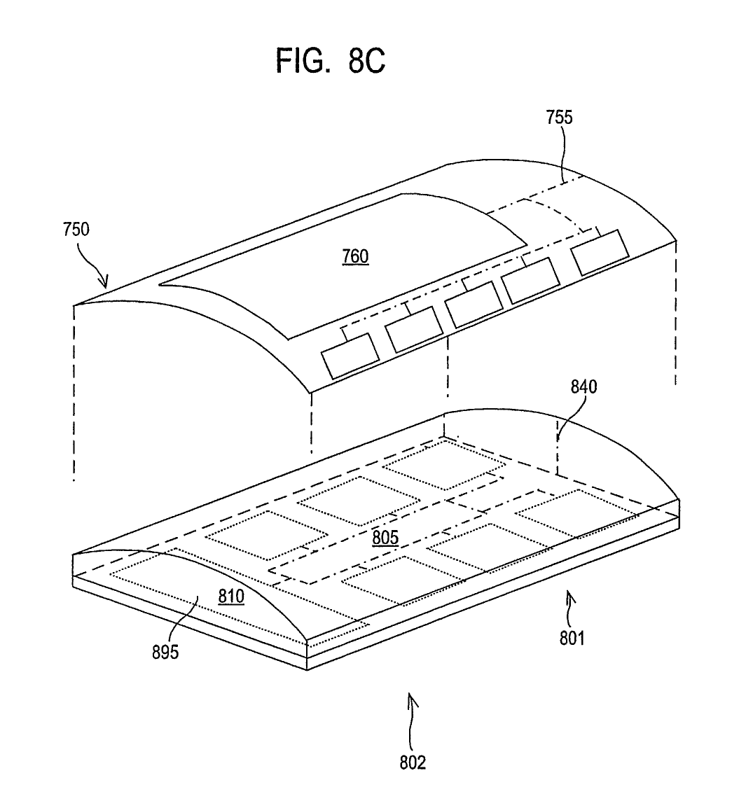

In certain embodiments, Applicants' ultrasound emitting device includes one or more diagnostic ultrasound emitters in combination with a plurality of therapeutic ultrasound emitters. In the illustrated embodiments of FIG. 8A, ultrasound emitting device 800 includes diagnostic ultrasound transceiver 810, and a 2×3 sound head matrix comprising 6 therapeutic ultrasound emitters. In other embodiments, Applicants' ultrasound emitting device comprises a plurality of diagnostic ultrasound transducers. In certain embodiments, one or more of the ultrasound transducers disposed in Applicants' ultrasound emitting device are capable of functioning as both a diagnostic ultrasound emitter and a therapeutic ultrasound emitter.

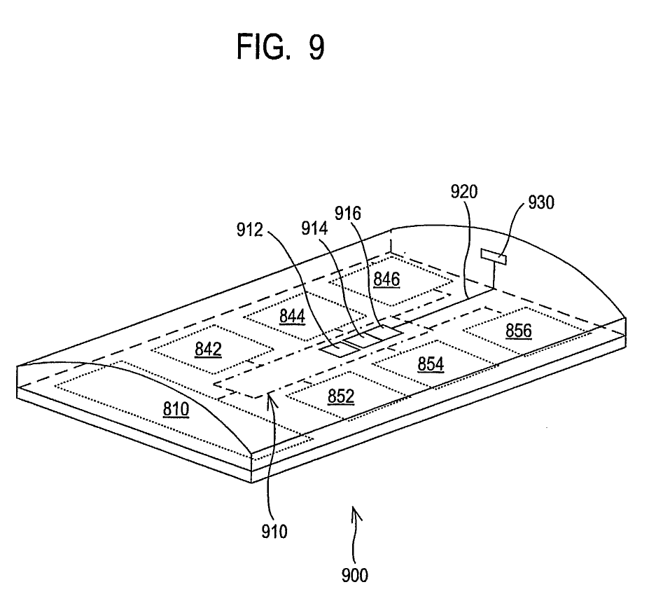

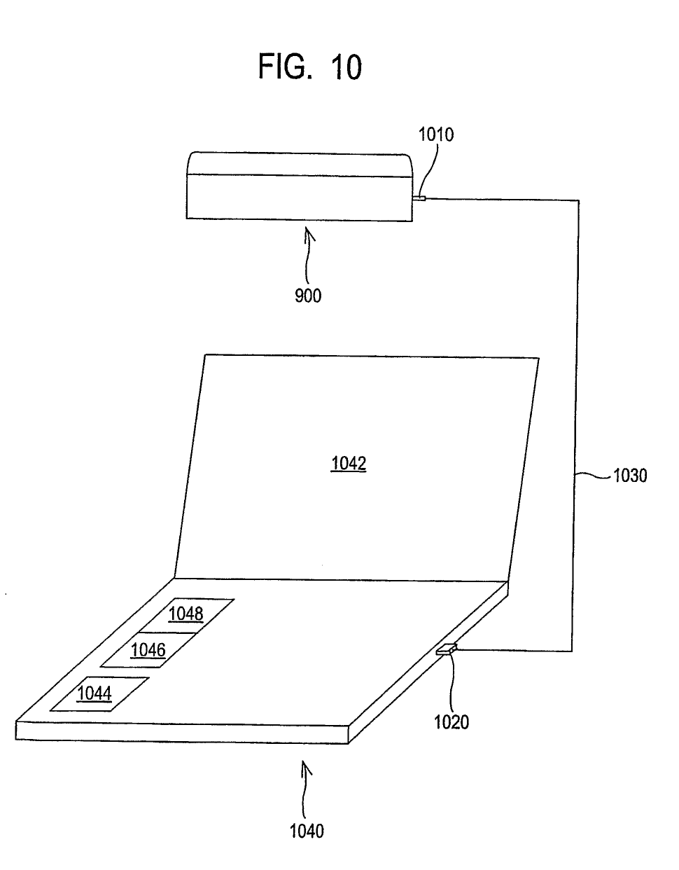

In the illustrated embodiment of FIG. 8A, ultrasound emitting device 800 comprises ultrasound transceiver 810 comprising diagnostic ultrasound emitter 812 and receiving device 814. By “diagnostic ultrasound emitter,” Applicants' mean a device which is capable of emitting diagnostic ultrasound energy having a output power of between about 0.5 and about 1 milliwatt per cm² at a frequency of between about 7 and about 13 megahertz. Emitter 812 produces and emits ultrasound waves. Receiver 814 detects emissions reflected back to transceiver 810 by various underlying body tissues. Those reflected emissions are processed by the controller, such as for example controller 620 (FIG. 6) and/or controller 720 (FIGS. 7A, 7B), and/or controller 805 (FIGS. 8B, 8C), and/or controller 910 (FIG. 9), and that controller causes a visual display device, such as visual display device 750 or visual display device 1042 (FIG. 10), to display an image of the tissue structure underlying the diagnostic ultrasound transceiver.

Any of the various types of diagnostic ultrasound imaging devices may be employed in the practice of the invention. Preferably, the transceiver 810 employs a resonant frequency (RF) spectral analyzer. Applicants' one or more diagnostic ultrasound transducers emit relatively low power level ultrasound waves. The various body tissues differentially reflect a portion of those sound waves. Applicants' diagnostic transceiver detects those reflected signals. An interconnected controller, external to or integral with the ultrasound emitting device, such as for example controller 620 (FIG. 6), 805 (FIG. 8B), 720 (FIGS. 7A, 7B), 910 (FIG. 9), or computing device 1040 (FIG. 10), processes those reflected signals and generates an image signal. That image signal is provided to a display device, external to or integral with the ultrasound emitting device, such as visual display device 760 (FIGS. 7B, 8C), or 1042 (FIG. 10), which visually displays an image of the tissues and structures underlying the ultrasound emitting device.

In certain embodiments, Applicants' apparatus and method employ harmonic imaging and/or pulse inversion imaging. In harmonic imaging, the bandwidth of the transmitted and received imaging signals must be narrow enough to ensure that the received harmonic signal can be separated from the transmitted fundamental signal.

Pulse inversion imaging avoids these bandwidth limitations and overcomes the contrast detectability and imaging resolution trade-off by using broader transmit and receive bandwidths. In pulse inversion imaging, a sequence of two ultrasound imaging pulses is transmitted into tissue instead of only a single pulse. The first pulse is an in-phase pulse, the second is an identical copy of the first, but inverted. For any linear target, the response to the second pulse is an inverted copy of the response from the first pulse. These are then summed and all linear echoes cancel.

On the other hand, for a nonlinear target, such as for example gas bubbles, the responses to positive and negative pulses differ. The addition of the responses does not cancel completely. Rather, the fundamental components of the echo cancel whereas the harmonic components add, giving twice the harmonic level of a single pulse. The main advantage of pulse inversion over harmonic imaging and harmonic power Doppler imaging is that it can function over the entire bandwidth of the received echo signal and, therefore, achieves superior imaging resolution.

In certain embodiments, Applicants' imaging method employs pulse inversion imaging using a low mechanical index (“MI”) thereby prolonging the lifetime of the contrast agent and obviating the need for intermittent imaging. In certain embodiments, Applicants' apparatus and method further employ a longer sequence of transmitted inverted pulses in order to remove tissue motion.

In still other embodiments, Applicants' imaging method utilize pulse inversion detection in combination with Doppler detection to exploit the advantages of both detection schemes. In these embodiments, more than two imaging pulses are transmitted and special Doppler filters are applied to remove tissue motion.

In yet other embodiments, Applicants' apparatus and method utilize power modulation for contrast agent detection based on nonlinear properties of gas micro bubbles. In these embodiments, Applicants' apparatus and method employ a multi-pulse technique wherein the acoustic amplitude of the transmitted imaging pulses is varied. For example, two transmit amplitudes are used, full and half amplitude. This transmit amplitude change induces changes in the response of the contrast agent. On receive, echoes from the half amplitude-transmitted pulse are adjusted in amplitude and subsequently subtracted from the full amplitude echoes. This procedure removes most of the linear responses at the fundamental frequency, and the remaining echoes contain mainly nonlinear signals from the micro bubbles.

In certain embodiments, Applicants' imaging method utilizes power modulation with a low-frequency wide band transducer. The low frequency transducer increases the depth of field and transmits the ultrasound energy more uniformly throughout the image. The combination of power modulation and wide band transducer allows ultraharmonic imaging, which results in a better elimination of tissue artifacts and therefore increased contrast to tissue ratio.

Referring once again to FIG. 8A, therapeutic ultrasound emitters 842, 844, and 846, are disposed on, or through, planar member 820. Emitters 842, 844, and 846, in combination with planar member 820, comprise planar assembly 860. Therapeutic ultrasound emitters 852, 854, 856, are disposed on, or through, planar member 830. Emitters 852, 854, and 856, in combination with planar member 830, comprise planar assembly 870.

Planar assembly 860 is continuously attached to planar assembly 870 along seam 825. In certain embodiments, the dihedral angle formed by the intersection of planar assembly 860 and planar assembly 870 is 180 degrees, i.e. the angle Φ shown in FIG. 8A is zero. In other embodiments, planar assembly 860 is offset from planar assembly 870, i.e. the angle Φ shown in FIG. 8A is greater than zero.

The illustrated embodiment of FIG. 8A comprises one embodiment of Applicants' ultrasound emitting device comprising both diagnostic and therapeutic ultrasound transducers. As a general matter, Applicants' ultrasound emitting device comprising both diagnostic and therapeutic transducers comprises a Y×Z sound head matrix, wherein Y represents the number of transducers in a column, and wherein Z represents the number of columns, wherein Y is greater than or equal to 1, and less than or equal to about 10, and wherein Z is greater than or equal to 1 and less than or equal to about 6. In certain embodiments, Applicants' diagnostic/therapeutic ultrasound emitting device comprises such a Y×Z therapeutic transducer sound head matrix in combination with one or more diagnostic transducers 812 and a receiver 814. In other embodiments, Applicants' diagnostic/therapeutic ultrasound emitting device comprises such a Y×Z therapeutic transducer sound head matrix in combination with receiver 814, wherein one or more of the therapeutic transducers is capable of emitting diagnostic ultrasound energy.

Referring now to FIG. 8B, Applicants' ultrasound emitting device 800 comprises sound head matrix assembly 801 in combination with controller 805 and housing 170. Controller 805 includes a processor such as processor 622, memory such as memory 624, and device microcode such as microcode 626, wherein processor 622 utilizes microcode 626 to operate the plurality of therapeutic emitters 842, 844, 846, 852, 854, and 856, and to operate diagnostic transducer 812, and to operate receiver 814.

In certain embodiments, Applicants' ultrasound device 800 includes an integral information input/output device. Referring now to FIG. 8C, ultrasound emitting device 802 comprises device 800 in combination with integrated I/O device 750. Controller 805 communicates with I/O device 750 via communication links 804 and 755. Diagnostic transceiver 810 is internally disposed within device 801 adjacent end 890. In these embodiments, controller 805 includes a processor, such as processor 622, memory, such as memory 624, and device microcode, such as microcode 626, to operate the plurality of therapeutic emitters 842, 844, 846, 852, 854, and 856, and to operate diagnostic transceiver 810, and to operate visual display device 760.

By monitoring display device 760, the medical provider can determine when sufficient injected microbubbles have reached the occlusion site. At that time, the medical provider than causes the plurality of therapeutic ultrasound emitters to produce ultrasound energy having a higher power level than the diagnostic power levels emitted by transceiver 810. Those higher power ultrasound energy causes the microbubbles to rupture. After the flow of the injected microbubbles ceases, the medical provider then discontinues emission of the therapeutic ultrasound energy.

In certain embodiments Applicants' ultrasound device includes an “auto-detect” feature, wherein that devices monitors the reflected diagnostic signals, and automatically detects the arrival of sufficient injected microbubbles at the occlusion site. When sufficient injected microbubbles are detected, Applicants' device automatically causes the plurality of therapeutic ultrasound devices to emit therapeutic ultrasound energy using a plurality of pre-determined therapeutic insonation regimes. When the flow of microbubbles ceases, Applicants' device automatically causes the plurality of therapeutic ultrasound devices to stop emitting therapeutic ultrasound energy.

In certain embodiments of Applicants' apparatus and method comprise “burst-mode” insonation embodiments, wherein in response to a detected event Applicants' ultrasound emitting device emits acoustic energy waves in bursts, using a plurality of pre-determined therapeutic insonation regimes, each such regime comprising a modulation pattern of duty cycles, frequencies, and phases. The period of insonation is followed by a period of no acoustic wave emissions. In certain embodiments, Applicants' burst mode insonation method comprises alternating a time period comprising bursts of acoustic energy followed by a time period of no acoustic energy emissions.

In certain embodiments, the detected event comprises a physiologic event. In other embodiments, the detected event comprises a non-physiologic event. Such a non-physiologic event comprises for example and without limitation a pre-determined time interval between the administration of one or more therapeutic agents and the initiation of acoustic energy emissions.

Such a detected physiologic event comprises for example and without limitation, a threshold heart rate, a threshold blood pressure, a threshold serum level of one or more compounds, and the like. In other embodiments, such an event comprises a non-detection event, for example the operation of Applicants' apparatus described herein is initiated upon imaging which shows the absence of a hemorrhagic stroke.

In certain embodiments, Applicants' controller/computing device 620, 720, 805/910, 1040, causes the plurality of therapeutic ultrasound transducers to emit acoustic waves, using a plurality of pre-determined therapeutic insonation regimes, in bursts, when a pre-determined concentration of microbubbles is detected. Each acoustic energy emission is followed by a period of no acoustic wave emissions. During the periods of no emissions, the concentration of microbubbles at the occlusion site is allowed to increase. When the pre-determined concentration of microbubbles is again detected, the controller again cause the plurality of ultrasound transducers to emit another burst of acoustic energy waves.

In certain embodiments, Applicants' ischemic stroke treatment protocol comprises selecting a sound head matrix comprising (N) therapeutic ultrasound transducers, establishing (N) therapeutic insonation regimes, wherein the (i)th therapeutic insonation regime is established for the (i)th therapeutic ultrasound transducer, wherein (N) is greater than or equal to 1, and wherein (i) is greater than or equal to 1 and less than or equal to (N). In certain embodiments, each (i)th therapeutic insonation regime comprises the (i)th duty cycle modulation pattern, the (i)th frequency modulation pattern, the (i)th power modulation pattern, and the (i)th phase modulation. In certain embodiments, selecting a sound head matrix and establishing the plurality of insonation regimes comprise selecting an ultrasound emitting device having a plurality of insonation regimes encoded to a processor disposed in the selected ultrasound emitting device.

In other embodiments, an insonation regime for each therapeutic ultrasound transducer disposed on the selected sound head matrix is created using a computing device external to the ultrasound emitting device comprising the selected sound head matrix. In certain of these embodiments, the external computing device remains interconnected to the ultrasound emitting device throughout Applicants' ischemic stroke treatment protocol, wherein the external computing device, using the pre-determined plurality of insonation regimes, controls the operation of each therapeutic transducer disposed on the selected sound head matrix. In other embodiments, the pre-determined plurality of insonation regimes is downloaded from the external computing device to a controller integral with the ultrasound emitting device comprising the selected sound head matrix.

In certain embodiments, Applicants' ischemic stroke treatment protocol further comprises establishing one or more imaging regimes. In certain embodiments, such imaging regimes utilize harmonic imaging. In certain embodiments, such imaging regimes utilize pulse inversion imaging. In certain embodiments, such imaging regimes utilize pulse inversion imaging using a low MI, In certain embodiments, such imaging regimes utilize pulse inversion imaging in combination with Doppler detection. In certain embodiments, such imaging regimes utilize power modulation. In certain embodiments, such imaging regimes utilize power modulation with a low-frequency wide band transducer.

In certain embodiments, establishing one or more imaging regimes comprise selecting an ultrasound emitting device having a one or more imaging regimes encoded in a processor disposed in the selected ultrasound emitting device. In other embodiments, an imaging regime is created using a computing device external to the ultrasound emitting device comprising the selected sound head matrix. In certain of these embodiments, the external computing device remains interconnected to the ultrasound emitting device throughout Applicants' ischemic stroke treatment protocol, wherein the external computing device, using the pre-determined imaging regimes, controls the operation of each diagnostic transducer disposed on the selected sound head matrix. In other embodiments, the pre-determined imaging regimes are downloaded from the external computing device to a controller integral with the ultrasound emitting device comprising the selected sound head matrix.

In the illustrated embodiment of FIG. 9, ultrasound energy emitting device 900 comprises a sound head matrix comprising plurality of therapeutic ultrasound transducers 842, 844, 846, 852, 854, 856, in combination with ultrasound transceiver 810, wherein controller 910 is in communication with each of the ultrasound transducers and with the ultrasound imaging transceiver 810. In certain embodiments, controller 910 comprises controller 805 (FIGS. 8B, 8C). As a general matter, ultrasound emitting device 900 comprises a sound head matrix assembly comprising a Y×Z sound head matrix, wherein Y represents the number of therapeutic transducers in a column, and wherein Z represents the number of columns, wherein Y is greater than or equal to 1, and less than or equal to about 10, and wherein Z is greater than or equal to 1 and less than or equal to about 6. In certain embodiments, one or more of the therapeutic transducers also comprises a diagnostic transducer.

In the illustrated embodiment of FIG. 9, controller 910 is interconnected with port 930 by communication link 920. In certain embodiments, port 930 comprises a Universal Serial Bus (“USB”) connection. In certain embodiments, port 930 comprises a USB 1.0 connection. In other embodiments, port 930 comprises a USB 2.0 connection. In certain embodiments, port 930 comprises an EEE 1394 compliant connection, sometimes referred to as a “firewire” connection.

In the illustrated embodiment of FIG. 9, controller 910 comprises processor element 912, memory element 914, and instructions/microcode 916 encoded to memory 914. In certain embodiments, controller 910 comprises an ASIC. Processor 912 utilizes instructions 916 to implement Applicants' ischemic stroke treatment protocol, wherein instructions 916 comprise a plurality of pre-determined therapeutic insonation regimes, and one or more pre-determined imaging regimes.

Referring now to FIG. 10, ultrasound emitting device 900 is interconnected with computing device 1040 via communication link 1030. Computing device 1040 comprises processor 1044, memory 1046, and instructions 1048. As a general matter, computing device 1040 comprises a computer system, such as a mainframe, personal computer, workstation, and combinations thereof, including an operating system such as Windows, AIX, Unix, MVS, LINUX, etc. (Windows is a registered trademark of Microsoft Corporation; AIX is a registered trademark and MVS is a trademark of IBM Corporation; UNIX is a registered trademark in the United States and other countries licensed exclusively through The Open Group; LINUX is a registered trademark owned by Linus Torvalds.)

Communication link 1030 is selected from the group comprising a wireless communication link, a serial interconnection, such as RS-232 or RS-422, an ethernet interconnection, a SCSI interconnection, an iSCSI interconnection, a Gigabit Ethernet interconnection, a Bluetooth interconnection, a Fibre Channel interconnection, an ESCON interconnection, a FICON interconnection, a Local Area Network (LAN), a private Wide Area Network (WAN), a public wide area network, Storage Area Network (SAN), Transmission Control Protocol/Internet Protocol (TCP/IP), the Internet, and combinations thereof.

In certain embodiments, a therapeutic insonation regime for each therapeutic transducer disposed on the selected sound head matrix is created using computing device 1040, wherein that plurality of therapeutic insonation regimes is encoded in memory 1046 as a portion of instructions 1048. In certain embodiments, one or more diagnostic imaging regimes for each diagnostic transducer disposed on the selected sound head matrix is created using computing device 1040, wherein that plurality of therapeutic insonation regimes is encoded in memory 1046 as a portion of instructions 1048.

In certain embodiments, computing device 1040 remains in communication with ultrasound emitting device 900 via communication link 1030 throughout all or a portion of Applicants' ischemic stroke treatment protocol. In other embodiments, instructions 1048 comprising a plurality of therapeutic insonation regimes, and optionally one or more imaging regimes, is downloaded to instructions 916 (FIG. 9) via communication link 1030, wherein communication link 1030 is disabled prior to initiating Applicants' ischemic stroke treatment protocol.

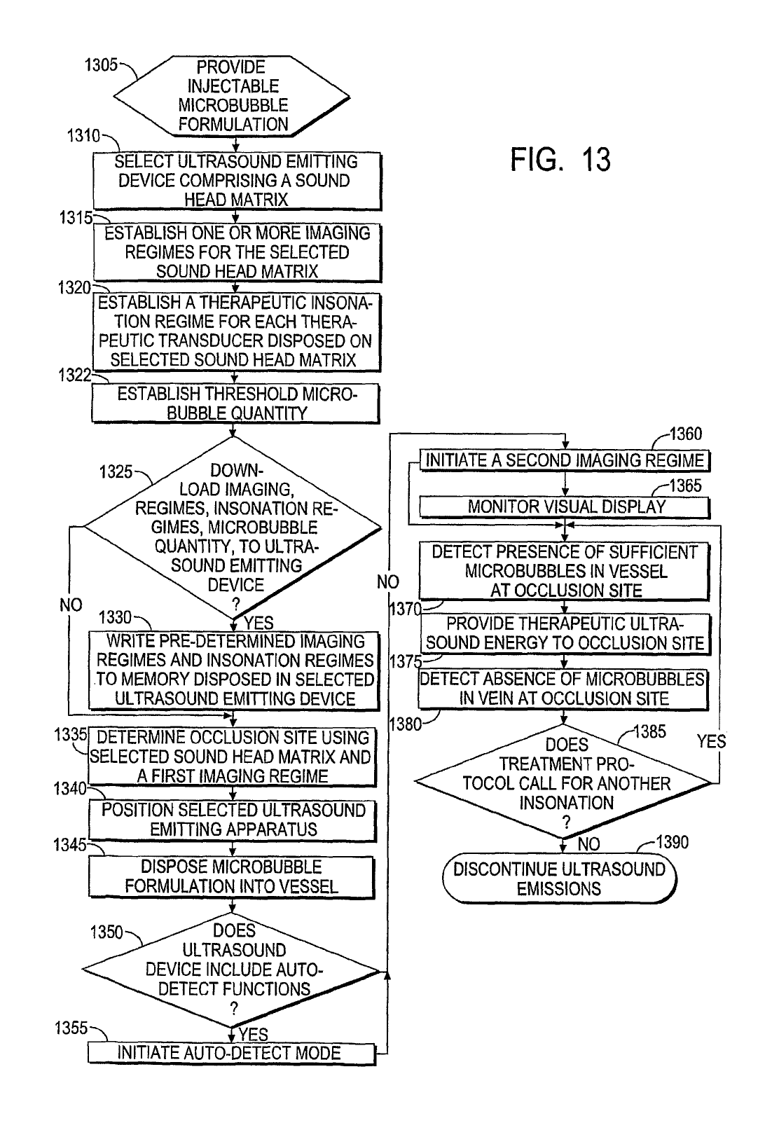

FIG. 13 summarizes Applicants' method to use the various embodiments of Applicants' ultrasonic device to treat an ischemic stroke, wherein an occluded vessel comprises an artery/vein disposed within a patient's cranial cavity.

In step 1305, the method provides an injectable microbubble formulation. U.S. Pat. Nos. 5,656,211 and 6,033,646 teach methods to form such a microbubble formulation, and are hereby incorporated by reference herein. U.S. Pat. No. 6,039,557 teaches an apparatus for preparing such a microbubble formulation, and is hereby incorporated by reference herein.

In step 1410, the method selects an ultrasound emitting apparatus comprising a sound head matrix comprising one or more diagnostic ultrasound transducers and one or more therapeutic ultrasound transducers, as described herein. In certain embodiments, the one or more diagnostic ultrasound transducers differ from the one or more therapeutic ultrasound transducers. In other embodiments, one or more ultrasound transducers function as both diagnostic transducers and therapeutic transducers. In certain embodiments, the ultrasound emitting device of step 1410 comprises Applicants' ultrasound emitting device 100 (FIGS. 1A, 1B). In certain embodiments, In certain embodiments, the ultrasound emitting device of step 1410 comprises Applicants' ultrasound emitting device 200 (FIGS. 2A, 4B). In certain embodiments, In certain embodiments, the ultrasound emitting device of step 1410 comprises Applicants' ultrasound emitting device 201 (FIG. 4C). In certain embodiments, In certain embodiments, the ultrasound emitting device of step 1410 comprises Applicants' ultrasound emitting device 300 (FIGS. 3A, 5B). In certain embodiments, In certain embodiments, the ultrasound emitting device of step 1410 comprises Applicants' ultrasound emitting device 301 (FIG. 5C). In certain embodiments, ultrasound emitting device 1120 comprises Applicants' ultrasound emitting device 600 (FIG. 6). In certain embodiments, In certain embodiments, the ultrasound emitting device of step 1410 comprises Applicants' ultrasound emitting device 710 (FIG. 7A). In certain embodiments, In certain embodiments, the ultrasound emitting device of step 1410 comprises Applicants' ultrasound emitting device 715 (FIG. 7B). In certain embodiments, In certain embodiments, the ultrasound emitting device of step 1410 comprises Applicants' ultrasound emitting device 800 (FIG. 8B). In certain embodiments, In certain embodiments, the ultrasound emitting device of step 1410 comprises Applicants' ultrasound emitting device 802 (FIG. 8C). In certain embodiments, In certain embodiments, the ultrasound emitting device of step 1410 comprises Applicants' ultrasound emitting device 900 (FIGS. 9, 10).

In step 1315, Applicants' method establishes one or more imaging regimes to be used by the one or more diagnostic transducers disposed on the sound head matrix selected in step 1310. In certain embodiments, such an imaging regime comprises utilizing harmonic imaging. In certain embodiments, such an imaging regime comprises utilizing pulse inversion imaging. In certain embodiments, such an imaging regime comprises utilizing pulse inversion imaging using a low MI, In certain embodiments, such an imaging regime comprises utilizing pulse inversion imaging in combination with Doppler detection. In certain embodiments, such an imaging regime comprises utilizing power modulation. In certain embodiments, such an imaging regime comprises utilizing power modulation with a low-frequency wide band transducer.

In step 1320, Applicants' method establishes a therapeutic insonation regime for each therapeutic transducer disposed on the sound head matrix selected in step 1310. In certain embodiments, such a plurality of therapeutic insonation regimes comprise a unique frequency modulation pattern and/or a unique phase modulation pattern, for each of the therapeutic ultrasound transducers. As described herein, in certain embodiments the acoustic waves emitted by the therapeutic ultrasound transducers comprising the selected sound head matrix are modulated such that, at any given time, none of those transducer are emitting acoustic waves having the same frequency and phase. In certain embodiments, certain of the therapeutic ultrasound transducers comprising the selected sound head matrix employ the “low to high” frequency modulation described above, while other transducers employ the “high to low” frequency modulation described herein.

In step 1325, Applicants' method determines whether to download the imaging regimes of step 1315 and the insonation regimes of step 1320 to the ultrasound emitting device selected in step 1310. If Applicants' method elects in step 1325 not to download one or more imaging regimes and a plurality of insonation regimes to the ultrasound emitting device, then Applicants' method transitions from step 1325 to step 1335, and an external controller, such as controller 620 (FIG. 1) or computing device 1040 (FIG. 10), controls the operation of the one or more diagnostic transducers and the plurality of therapeutic transducers disposed within the selected ultrasound emitting device selected in step 1310.

Alternatively, if Applicants' method elects in step 1325 to download one or more imaging regimes and a plurality of insonation regimes to the ultrasound emitting device, then Applicants' method transitions from step 1325 to step 1330 wherein the method downloads to the ultrasound emitting device selected in step 1310 the one or more imaging regimes established in step 1315 and the plurality of therapeutic insonation regimes established in step 1320.

Applicants' method transitions from step 1330 to step 1335 therein Applicants' method locates the occlusion site using the selected ultrasound emitting device comprising the selected sound head matrix and one or more of the imaging regimes of step 1315. In certain embodiments, step 1335 comprises positioning the selected ultrasound emitting device on the patients' scalp by hand.

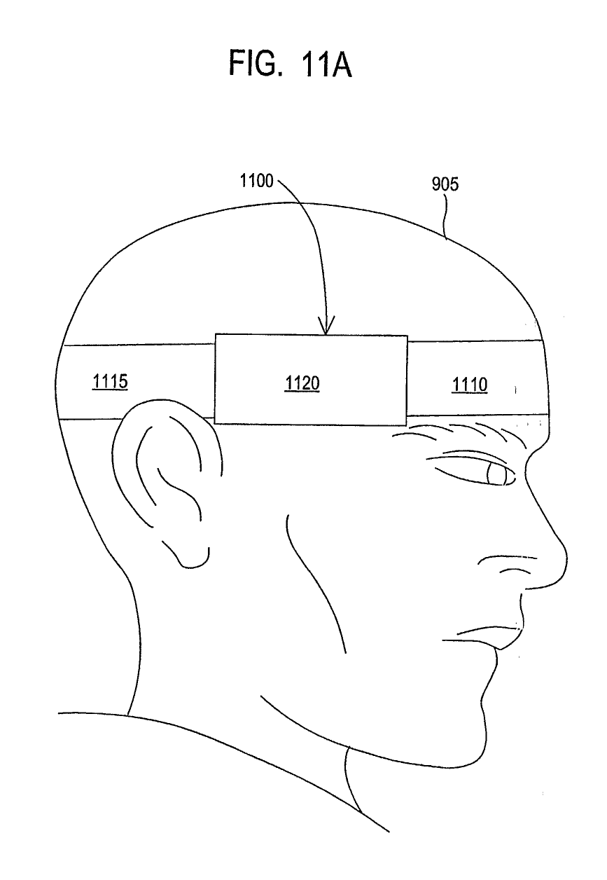

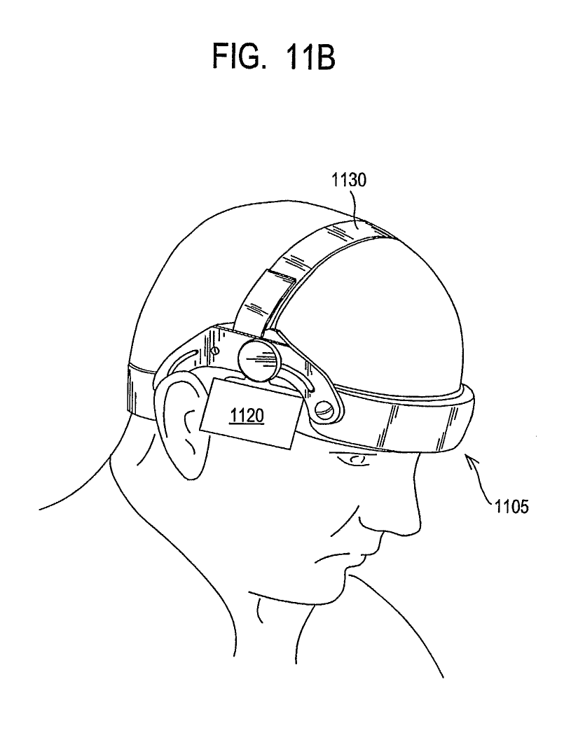

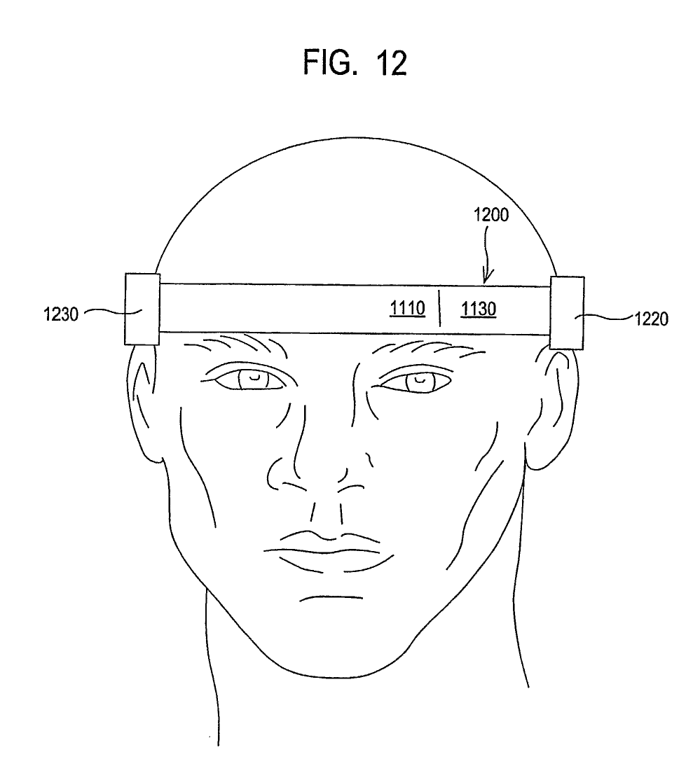

Referring now to FIGS. 11A, 11B, and 12, in other embodiments step 1335 comprises utilizing apparatus 1100 (FIG. 11A), or apparatus 1105 (FIG. 11B), or apparatus 1200 (FIG. 12). In the illustrated embodiment of FIG. 11A, apparatus 1100 comprises ultrasound emitting device 1120 in combination with head band elements 1110 and 1115. In certain embodiments, head band portions 1110 and 1115 comprise an integral assembly which can be disposed circumferentially around a patient's head. In certain embodiments, head band portions 1110 and 1115 comprise an elastic material which can be stretched in order to place assembly 1100 around the head, and which then contracts to hold assembly 1100 in place around the head.

Applicants have found that insonation of the basal cerebral arteries and the circle of Willis is facilitated by placing Applicants' ultrasound emitting assembly 1100 around head such that the acoustic wave(s) emitted by ultrasound emitting device 1120 cross the thinnest portion of the squamous part of the temporal bone. The temporal window can be localized quite anteriorly (close to the vertical portion of the zygomatic bone) or, more frequently, posteriorly (close to the pinna of the ear).

In certain embodiments, ultrasound emitting device 1120 comprises Applicants' ultrasound emitting device 100 (FIGS. 1A, 1B). In certain embodiments, ultrasound emitting device 1120 comprises Applicants' ultrasound emitting device 200 (FIGS. 2A, 4B). In certain embodiments, ultrasound emitting device 1120 comprises Applicants' ultrasound emitting device 201 (FIG. 4C). In certain embodiments, ultrasound emitting device 920 comprises Applicants' ultrasound emitting device 300 (FIGS. 3A, 5B). In certain embodiments, ultrasound emitting device 1120 comprises Applicants' ultrasound emitting device 301 (FIG. 5C). In certain embodiments, ultrasound emitting device 1120 comprises Applicants' ultrasound emitting device 600 (FIG. 6). In certain embodiments, ultrasound emitting device 1120 comprises Applicants' ultrasound emitting device 710 (FIG. 7A). In certain embodiments, ultrasound emitting device 1120 comprises Applicants' ultrasound emitting device 715 (FIG. 7B). In certain embodiments, ultrasound emitting device 1120 comprises Applicants' ultrasound emitting device 800 (FIG. 8B). In certain embodiments, ultrasound emitting device 1120 comprises Applicants' ultrasound emitting device 802 (FIG. 8C). In certain embodiments, ultrasound emitting device 1120 comprises Applicants' ultrasound emitting device 900 (FIGS. 9, 10).

Referring now to FIG. 11B, apparatus 1105 comprises one or more ultrasound emitting devices 1120, as described hereinabove, attached to head frame 1130. In the illustrated embodiment of FIG. 12, Applicants' ultrasound emitting assembly 1200 comprises ultrasound emitting device 1220 and ultrasound emitting device 1230. Devices 1220 and 1230 are attached to opposing sides of head band portion 1110 (FIGS. 11, 12), or to opposing sides of head frame 1130. In certain embodiments, one or more of ultrasound emitting devices 1220 and 1230 comprise Applicants' ultrasound emitting device 100 (FIGS. 1A, 1B). In certain embodiments, one or more of ultrasound emitting devices 1220 and 1230 comprise Applicants' ultrasound emitting device 200 (FIGS. 2A, 4B). In certain embodiments, one or more of ultrasound emitting devices 1220 and 1230 comprise Applicants' ultrasound emitting device 201 (FIG. 4C). In certain embodiments, u one or more of ultrasound emitting devices 1220 and 1230 comprise Applicants' ultrasound emitting device 300 (FIGS. 3A, 5B). In certain embodiments, u one or more of ultrasound emitting devices 1220 and 1230 comprise Applicants' ultrasound emitting device 301 (FIG. 5C). In certain embodiments, one or more of ultrasound emitting devices 1220 and 1230 comprise Applicants' ultrasound emitting device 600 (FIG. 6). In certain embodiments, one or more of ultrasound emitting devices 1220 and 1230 comprise Applicants' ultrasound emitting device 710 (FIG. 7A). In certain embodiments, one or more of ultrasound emitting devices 1220 and 1230 comprise Applicants' ultrasound emitting device 715 (FIG. 7B).). In certain embodiments, u one or more of ultrasound emitting devices 1220 and 1230 comprise Applicants' ultrasound emitting device 800 (FIG. 8B). In certain embodiments, one or more of ultrasound emitting devices 1220 and 1230 comprise Applicants' ultrasound emitting device 802 (FIG. 8C). In certain embodiments, one or more of ultrasound emitting devices 1220 and 1230 comprise Applicants' ultrasound emitting device 900 (FIGS. 9, 10).

Referring once again to FIG. 13, having located the occlusion site in step 1335 in step 1340 Applicants' method positions the ultrasound emitting device selected in step 1310 on the patient's scalp such that the ultrasound energy waves emitted from the plurality of therapeutic transducers disposed on the selected sound head matrix are directed to the occlusion site. In certain embodiments, step 1340 comprises positioning the ultrasound emitting device of step 1310 by hand. In other embodiments, step 1340 comprises positioning one or more ultrasound emitting devices of step 1310 using the head band apparatus 1100 (FIG. 11A) or head frame apparatus 1105 (FIG. 11B).

In step 1345, a medical provider causes the microbubble formulation of step 1305 to be disposed into the occluded vessel distal to the occlusion site. In step 1350, Applicants' method determines if the selected ultrasound emitting device comprises an auto-detect function. If Applicants' method determines in step 1350 that the selected ultrasound emitting device does not comprise an auto-detect function, then the method transitions from step 1350 to step 1360.

If Applicants' method determines in step 1350 that the device selected in step 1310 comprises one or more diagnostic transducers, a receiver, and an auto-detect function, then Applicants' method transitions from step 1350 to step 1355 wherein the operator initiates the auto-detect function. In embodiments wherein the selected device includes one or more diagnostic ultrasound transducers, a receiver, and an auto-detect function, the operator need do no more than initiate the auto-detect function. The ultrasound emitting apparatus then automatically detects the arrival of sufficient microbubbles at the occlusion site, automatically implements the pre-determined therapeutic insonation regimes, automatically detects the absence of microbubbles at the occlusion site, and automatically discontinues ultrasound emissions. In certain embodiments, Applicants' auto-detect function implements, inter alia, Applicants' burst-mode insonation treatment regime as described herein.

Applicants' method transitions from step 1355 to step 1360 wherein Applicants' method initiates one or more of the imaging regimes of step 1315. In certain embodiments, step 1360 is performed by a controller external to, or integral with, the ultrasound emitting device selected in step 1310. In other embodiments, step 1360 is performed by medical personnel.

In certain embodiments, steps 1335 and 1360 utilize the same one or more imaging regimes. In other embodiments, steps 1335 and 1360 utilize different imaging regimes. In step 1365, the operator monitors the operable visual display device. In certain embodiments, the visual display device of step 1365, such as visual display device 1042 (FIG. 10) disposed in computing device 1040, is external to the ultrasound emitting device selected in step 1310. In other embodiments, the visual display device of step 1365, such as visual display device 760 (FIG. 8C), is integral with the ultrasound emitting device selected in step 1310.

In certain embodiments, Applicants' method transitions from step 1360 to step 1370. In other embodiments, Applicants' method includes step 1365 wherein an operator visually monitors a display device external to, or integral with, the ultrasound emitting device of step 1310. Applicants' method transitions from step 1365 to step 1370 wherein the method detects the presence of sufficient microbubbles at the occlusion site. Applicants have found that insonation of less than that minimum quantity of microbubbles results in less than optimal lysing effects.

In certain embodiments, step 1370 comprises determining if the quantity of microbubbles adjacent the occlusion site exceeds the microbubble threshold of step 1322. In certain embodiments, step 1370 is performed by a controller external to, or integral with, the ultrasound emitting device selected in step 1310. In other embodiments, step 1370 is performed by medical personnel.

Applicants' method transitions from step 1370 to step 1375 wherein the method causes the ultrasound device to provide therapeutic ultrasound energy to the occlusion site, using the plurality of therapeutic insonation regimes of step 1320. In certain embodiments, step 1375 is performed by a controller external to, or integral with, the ultrasound emitting device selected in step 1310. In other embodiments, step 1375 is performed by medical personnel.

In certain embodiments, in step 1375 a controller external to, or integral with, the selected ultrasound emitting device of step 1310 comprising (N) therapeutic ultrasound transducers, using the (i)th therapeutic insonation regime encoded in memory disposed in that controller, wherein the (i)th therapeutic insonation regime comprises a duty cycle, the (i)th power modulation pattern, the (i)th frequency modulation pattern, and the (i)th phase modulation pattern, provides the (i)th signal to the (i)th therapeutic ultrasound transducer thereby causing that (i)th transducer to emit therapeutic ultrasound energy.

In step 1380, the method determines the absence of microbubbles at the occlusion site, and discontinues ultrasound emissions. In certain embodiments, step 1380 is performed by a controller external to, or integral with, the ultrasound emitting device selected in step 1310. In other embodiments, step 1380 is performed by medical personnel.

Applicants' method transitions from step 1380 to step 1385 wherein the method determines if the plurality of therapeutic insonation regimes of step 1320 comprises multiple insonations. In certain embodiments, step 1385 is performed by a controller external to, or integral with, the ultrasound emitting device selected in step 1310. In other embodiments, step 1385 is performed by medical personnel.

If Applicants' method determines in step 1385 that the plurality of therapeutic insonation regimes of step 1320 comprises Applicants' burst-mode insonation embodiment, then the method transitions from step 1385 to step 1365 and continues as described herein. Alternatively, if Applicants' method determines in step 1385 that Applicants' ischemic stroke treatment protocol does not require an additional insonation, then the method transitions from step 1385 to step 1390 wherein the method discontinues the treatment protocol.

In certain embodiments, individual steps recited in FIG. 13, may be combined, eliminated, or reordered.

In certain embodiments, Applicants' invention includes microcode, such as microcode 626, instructions 916, and/or instructions 1048, wherein the microcode/instructions are executed by a processor, such as 622 (FIG. 6), 912 (FIG. 9), 1044 (FIG. 10), respectively, to perform one or more of steps 1335, 1360, 1370, 1375, 1380, 1385, and/or 1390, recited in FIG. 13.

In other embodiments, Applicants' invention includes instructions residing in any other computer program product, where those instructions are executed by a computer external to, or internal to, Applicants' apparatus to perform steps one or more of steps 1335, 1360, 1370, 1375, 1380, 1385, and/or 1390, recited in FIG. 13. In either case, the microcode/instructions may be encoded in an information storage medium comprising, for example, a magnetic information storage medium, an optical information storage medium, an electronic information storage medium, and the like. By “electronic storage media,” Applicants mean, for example, a device such as a PROM, EPROM, EEPROM, Flash PROM, compactflash, smartmedia, and the like.

While the preferred embodiments of the present invention have been illustrated in detail, it should be apparent that modifications and adaptations to those embodiments may occur to one skilled in the art without departing from the scope of the present invention.

We claim:

1. A method for treating a patient sustaining cerebral ischemia or an ischemic stroke in the brain, comprising the steps of:
supplying an ultrasound emitting device comprising a plurality of ultrasound transducers;
emitting first ultrasound energy by said ultrasound emitting device, wherein said first ultrasound energy comprises first power;
locating using said first ultrasound energy an occlusion disposed in one of said patient's cerebral blood vessels,
emitting second ultrasound energy by said ultrasound emitting device, wherein said second ultrasound energy comprises second power, wherein said second power is greater than said first power;
lysing said occlusion using said second ultrasound energy.

2. The method of claim 1, wherein said supplying an ultrasound emitting device step further comprises supplying an ultrasound emitting device comprising (N) therapeutic ultrasound transducers capable of emitting said second ultrasound energy, wherein (N) is greater than 1, said method further comprising the steps of:
supplying a controller interconnected with each of said (N) ultrasound transducers;
providing by said controller the (i)th signal to the (i)th therapeutic ultrasound transducer, wherein (i) is greater than or equal to 1 and less than or equal to (N);
wherein said emitting second ultrasound energy step further comprises emitting by the (i)th therapeutic ultrasound transducer the (i)th second ultrasound energy comprising the (i)th frequency and the (i)th phase.

3. The method of claim 2, further comprising the steps of:
establishing (N) therapeutic insonation regimes, wherein the (i)th therapeutic insonation regime comprises the (i)th frequency pattern and the (i)th phase pattern;
wherein said supplying a controller step further comprises supplying a controller comprising a processor and memory;
encoding said (N) therapeutic insonation regimes in said memory;
using by said processor the (i)th therapeutic insonation regime to generate said (i)th signal.

4. The method of claim 3, wherein said supplying an ultrasound emitting device further comprises supplying an ultrasound emitting device comprising said controller.

5. The method of claim 4, further comprising the steps of:
supplying a computing device;
wherein said establishing (N) therapeutic insonation regimes is performed using said computing device;
downloading said (N) therapeutic insonation regimes from said computing device to said memory.

6. The method of claim 3, wherein the (i)th frequency pattern differs from the frequency pattern associated each of the remaining (N−1) therapeutic insonation regimes.

7. The method of claim 3, wherein the (i)th phase pattern differs from the phase pattern associated each of the remaining (N−1) therapeutic insonation regimes.

8. The method of claim 1, wherein said supplying an ultrasound emitting device step further comprises supplying an ultrasound emitting device comprising a diagnostic ultrasound transducer capable of emitting said first ultrasound energy, a receiver to detect reflected ultrasound energy;
supplying a controller interconnected with said diagnostic ultrasound transducer and said receiver;
supplying a visual display device interconnected with said controller;
receiving reflected first ultrasound energy;
generating by said controller an image of the tissue structure underlying said diagnostic ultrasound transceiver;
displaying said image on said visual display device.

9. The method of claim 8, further comprising the steps of:
establishing an imaging regime;
wherein said supplying a controller step further comprises supplying a controller comprising a processor and memory;
encoding said imaging regime in said memory;
using by said processor said imaging regime to cause said diagnostic ultrasound transducer to emit said first ultrasound energy.

10. The method of claim 9, wherein said establishing an imaging regime, further comprises establishing an imaging regime selected from the group consisting of harmonic imaging, pulse inversion imaging, pulse inversion imaging using a low mechanical index, pulse inversion imaging in combination with Doppler detection, and power modulation imaging.

11. The method of claim 8, wherein said supplying an ultrasound emitting device further comprises supplying an ultrasound emitting device comprising said controller.

12. The method of claim 11, wherein said supplying an ultrasound emitting device further comprises supplying an ultrasound emitting device comprising said visual display device.

13. The method of claim 11, further comprising the steps of:
supplying a computing device;
wherein said establishing an imaging regime is performed using said computing device;
downloading said imaging regime from said computing device to said memory.

14. The method of claim 8, further comprising the steps of:
providing a mixture comprising a plurality of gas-filled microbubbles;
providing a therapeutically effective amount of said mixture in said occluded cerebral vessel;
detecting said gas-filled microbubbles adjacent said occlusion prior to emitting said second ultrasound energy.

15. The method of claim 14, wherein said detecting gas-filled microbubbles step further comprises:
establishing a threshold quantity of microbubbles;
determining if the detected microbubbles adjacent said occlusion exceed said threshold quantity;
operative if the detected microbubbles adjacent said occlusion exceed said threshold quantity, emitting said second ultrasound energy.

16. An article of manufacture comprising at least one diagnostic ultrasound transducer to emit first ultrasound energy comprising first power, (N) therapeutic ultrasound transducers to emit second ultrasound energy comprising second power, and a computer readable medium having computer readable program code disposed therein to operate said article of manufacture, wherein (N) is greater than or equal to 1, and wherein said second power is greater than said first power, the computer readable program code comprising a series of computer readable program steps to effect:
providing the (i)th signal to the (i)th therapeutic ultrasound transducer, wherein (i) is greater than or equal to 1 and less than or equal to (N);
emitting by said (i)th therapeutic ultrasound transducer second ultrasound energy comprising the (i)th frequency and the (i)th phase.

17. The article of manufacture of claim 16, wherein said article of manufacture further comprises memory and (N) therapeutic insonation regimes encoded in said memory, wherein the (i)th therapeutic insonation regime comprises the (i)th frequency pattern and the (i)th phase pattern, and wherein, for each value of (i), the (i)th frequency pattern differs from the frequency pattern comprised by each of the remaining (N−1) therapeutic insonation regimes.

18. The article of manufacture of claim 17, wherein, for each value of (i), the (i)th phase pattern differs from the phase pattern comprised by each of the remaining (N−1) therapeutic insonation regimes.

19. The article of manufacture of claim 18, further comprising a receiver to detect reflected first ultrasound energy and a visual display device, said computer readable program code further comprising a series of computer readable program steps to effect:
emitting first ultrasound energy;
receiving reflected first ultrasound energy;
generating an image of the tissue structure underlying said diagnostic ultrasound transducer;
displaying on said visual display said device.

20. The article of manufacture of claim 19, wherein said article of manufacture further comprises a threshold quantity of microbubbles, said computer readable program code further comprising a series of computer readable program steps to effect:
detecting gas-filled microbubbles adjacent an occlusion in a blood vessel;
determining if the detected microbubbles adjacent said occlusion exceed said threshold quantity;
operative if the detected microbubbles adjacent said occlusion exceed said threshold quantity, emitting said second ultrasound energy.
